(19) United States
(12) Patent Application Publication
(10) Pub. No.: US 2006/0160906 A1
(43) Pub. Date: Jul. 20, 2006

(54) Polyamine analogs as therapeutic agents for ocular diseases

(76) Inventors: Laurence J. Marton, Palo Alto, CA (US); Sterling Haidt, Mountain View, CA (US); Peter Campochiaro, Baltimore, MD (US)

Correspondence Address: MORRISON & FOERSTER LLP, 755 PAGE MILL RD, PALO ALTO, CA, 94304-1018, US

(21) Appl. No.: 11/244,095

(22) Filed: Oct. 4, 2005

Related U.S. Application Data
(60) Provisional application No. 60/616,089, filed on Oct. 4, 2004.
(60) Provisional application No. 60/676,638, filed on Apr. 29, 2005.

(51) Int. Cl. A61K 31/13

(52) U.S. Cl. 514/674

(57) ABSTRACT

This disclosure relates to methods of treating ocular diseases using polyamine analogs, particularly conformationally restricted polyamine analogs. The ocular diseases to be treated include a variety of ophthalmic disorders characterized by angiogenesis and/or neovascularization, including macular degeneration. Both wet macular degeneration and dry macular degeneration can be treated using the methods of the invention. The invention also provides ophthalmic formulations, including sustained release formulations and sustained release devices.

### CROSS REFERENCE TO RELATED APPLICATIONS

This application claims priority benefit of U.S. provisional patent application no. 60/616,089, filed Oct. 4, 2004, and of U.S. provisional patent application no. 60/676,638, filed Apr. 29, 2005. The entire contents of those applications are hereby incorporated by reference herein.

### STATEMENT REGARDING FEDERALLY SPONSORED RESEARCH OR DEVELOPMENT

This invention was made with government support under Grant Nos. EY05951, EY12609, and P30EY1765 awarded by the National Eye Institute. The Government has certain rights in the invention.

### TECHNICAL FIELD

This application relates to methods of treating ocular diseases, such as macular degeneration, using polyamine analogs, particularly conformationally restricted polyamine analogs.

### BACKGROUND

Age-related macular degeneration (AMD) is a leading cause of blindness in industrialized nations. The macula is the central portion of the retina, responsible for finely focused vision. The portion of the retina outside the macula is called the peripheral retina, and is responsible for peripheral vision. While the macula accounts for only a small portion of the total retina, the macula enables humans to read, recognize faces and other images, and perform other tasks requiring perception of detail. Thus macular degeneration can cause a devastating loss of visual ability.

Macular degeneration is typically classified as either “dry” macular degeneration or “wet” macular degeneration. Dry macular degeneration accounts for most cases of macular degeneration, and is usually less severe than wet macular degeneration. Dry macular degeneration is typically characterized by the formation of drusen. Drusen are deposits which form in the eye between the retinal pigment epithelium (RPE) and Bruch's membrane. The RPE and Bruch's membrane are structures which provide support to the retina and enable nutrients to reach the retina; abnormalities in these structures can lead to atrophy and death of the retinal cells responsible for vision. While the composition of drusen is not completely known, amyloid-beta has been found in drusen, and it is speculated that this amyloid-beta causes increased inflammation and oxidative stress (see Dentchev et al., Molecular Vision 9: 184-190 (2003)). Vision loss due to dry macular degeneration tends to be relatively gradual.

Wet macular degeneration accounts for only about 10% of macular degeneration cases. However, it can be much more severe and can occur much more rapidly than dry macular degeneration. Wet macular degeneration occurs when new blood vessels begin forming (a process known as neovascularization) behind the macula; this typically occurs in the region near drusen deposits, and appears to involve the breakdown of Bruch's membrane. These newly-formed blood vessels tend to leak, causing retinal detachment and scarring, which results in severe damage to the macula. [0007] Several treatments have been proposed for macular degeneration. One such treatment uses agents which inhibit neovascularization. Inhibition of the activity of vascular endothelial growth factor (VEGF, a factor involved in angiogenesis and neovascularization), has been proposed by several researchers in order to prevent neovascularization; see, e.g., U.S. Pat. No. 6,676,941. Another treatment for macular degeneration uses phototherapy, in which a photoactivated compound is administered to the patient. The compound can then be activated by light to selectively treat the abnormal blood vessels; see, e.g., U.S. Pat. No. 6,622,729. Visudyne (verteporfin) is a drug currently in use for photodynamic therapy; see, e.g., U.S. Pat. No. 4,883,79. The use of radiation therapy to treat neovascularization has been proposed as well (Flaxel, Ophthalmol Clin North Am. 15: 437-44 (2002)).

Other publications include WO 02/43722, directed to methods for treating ocular inflammation using copper chelating compounds, such as compounds other than D-penicillamine; in some embodiments, such compounds may be polyamines, such as triethylenetetramine or tetraethylenepentamine. WO 01/68053 is directed to methods and compositions for the prophylactic and therapeutic treatment of ophthalmic disorders associated with the posterior segment of the eye using topical ophthalmic compositions comprising therapeutic agents, which can be a polyamine. WO 2004/058289 is directed to an ophthalmic formulation for the prevention and treatment of adverse ocular conditions, including presbyopia, arcus senilis, age-related macular degeneration, and other conditions associated with aging. The application mentions polyamines such as cyclam (1,4,7,11-tetraazacyclotetradecane).

Prevention of macular degeneration would also be of great utility to patients at risk for the disease. Nutritional supplementation—consumption of higher than average levels of antioxidant vitamins and zinc—may lower the risk of macular degeneration. See, e.g., Sackett et al., Insight, 27: 5-7 (2002). Cigarette smoking is strongly associated with macular degeneration and other eye diseases (see, e.g., Cheng et al., Hong Kong Med J. 6: 195-202 (2000)), and smoking cessation is highly advisable for this and other health reasons. A report of an association of a specific single nucleotide polymorphism with macular degeneration may help identify individuals at risk for AMD (see Science 308: 385-9 (2005)). Other genes and mutations have also been identified which can help indicate those individuals at increased risk of macular degeneration.

Despite these efforts, macular degeneration continues to affect millions of people. Thus, new treatments are needed for this disease. Conformationally restricted polyamines are proposed for use in suppression and regression of choroidal neovascularization; see Silva et al., “Polyamine analogs suppress choroidal neovascularization (CNV),” Investigative Ophthalmology & Visual Science 45: U730 (2004), and Silva et al., “Suppression and regression of choroidal neovascularization by polyamine analogues” Investigative Ophthalmology & Visual Science 46: 3323 (2005), which discuss the compounds CGC-11144 and CGC-11150. See also Haidt et al., “Evidence for systemic immune activation in patients with ARMD,” Investigative Ophthalmology & Visual Science 45: U64 (2004); WO 99/21542; and US 2005/0159493.

Conformationally-restricted polyamine analogs and methods of synthesizing such analogs have been disclosed in U.S. Pat. Nos. 5,889,061, 6,392,098, and 6,794,545, U.S. Patent Application Publication Nos. 2003/0072715, 2003/0195377, and International Patent Applications WO 98/17624, WO 00/66587, WO 02/10142, and (WO 03/050072. These compounds have been shown to have anti-cancer effects in vitro or in vivo.

The instant application relates to the use of polyamines and polyamine analogs, such as conformationally-restricted polyamine analogs, for the treatment of ocular diseases, such as macular degeneration (both dry and wet forms of the disease).

### DISCLOSURE OF THE INVENTION

The present invention relates to methods of treating ocular diseases with polyamine analogs, such as conformationally restricted polyamine analogs. The methods of the invention embrace the use of polyamine analogs and compositions comprising a polyamine analog for treating ocular diseases. In one embodiment, the ocular disease is characterized by undesirable cell proliferation or neovascularization. The diseases include, but are not limited to, diseases caused by retinal or choroidal neovascularization, such as macular degeneration. In one embodiment, the polyamine analog(s) is conformationally restricted, and the ocular disease to be treated is macular degeneration. In another embodiment, the ocular disease is wet macular degeneration. In another embodiment, the ocular disease is dry macular degeneration. In another embodiment, the undesirable cell proliferation excludes cancer or other malignancies.

In one embodiment, the only conformational restriction of the polyamine analog is due to a carbon-carbon double bond (an ethenyl group, C═C) in the molecule; in this embodiment, a proviso is added to any or all of the embodiments below that the only conformational restriction of the polyamine analog is due to a carbon-carbon double bond. In another embodiment, the only conformational restriction of the polyamine analog is due to a cycloalkyl group, such as a cyclopropyl group, in the molecule; in this embodiment, a proviso is added to any or all of the embodiments below that the only conformational restriction of the polyamine analog is due to a cycloalkyl group, or due to a cyclopropyl group. In another embodiment, the conformationally restricted polyamine analog is not a macrocyclic polyamine analog; that is, the conformational restriction of the nitrogen does not arise from occurrence of two or more nitrogens in a cycle or macrocycle; in this embodiment, a proviso is added to any or all of the embodiments below that the conformationally restricted polyamine analog is not a macrocyclic polyamine analog. In one embodiment of the invention, a proviso is added that the compounds CGC-11144 and CGC-11150 are not included in the group of conformationally restricted polyamine analogs; in this embodiment, a proviso is added to any or all of the embodiments below that the conformationally restricted polyamine analog is not CGC-11144 nor CGC-11150. In another embodiment of the invention, a proviso is added that conformationally restricted polyamine analogs having ten nitrogens are not included in the group of conformationally restricted polyamine analogs; in this embodiment, a proviso is added to any or all of the embodiments below that the conformationally restricted polyamine analog is not a conformationally restricted polyamine analog having ten nitrogens. In another embodiment of the invention, a proviso is added that conformationally restricted polyamine analogs having ten nitrogens and a conformational restriction arising from a carbon-carbon double bond are not included in the group of conformationally restricted polyamine analogs; in this embodiment, a proviso is added to any or all of the embodiments below that the conformationally restricted polyamine analog is not a conformationally restricted polyamine analog having ten nitrogens and a conformational restriction arising from a carbon-carbon double bond.

In one embodiment, the conformationally restricted polyamine analog is selected from among compounds of the formula:
E-NH—B-A-B—NH—B-A-B—NH—B-A-B—NH—B-A-B—NH-E
where A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of: a single bond, C₁-C₆ alkyl, and C₂-C₆ alkenyl; and E is independently selected from the group consisting of H, C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; with the proviso that either at least one A moiety is selected from the group consisting of C₂-C₆ alkenyl, C₂-C₆ alkynyl, C₃-C₆ cycloalkyl, C₃-C₆ cycloaryl, and C₃-C₆ cycloalkenyl, or at least one B moiety is selected from the group consisting of C₂-C₆ alkenyl; and all salts, hydrates, solvates, and stereoisomers thereof. In one embodiment, the only conformational restriction of the polyamine analog is due to a carbon-carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 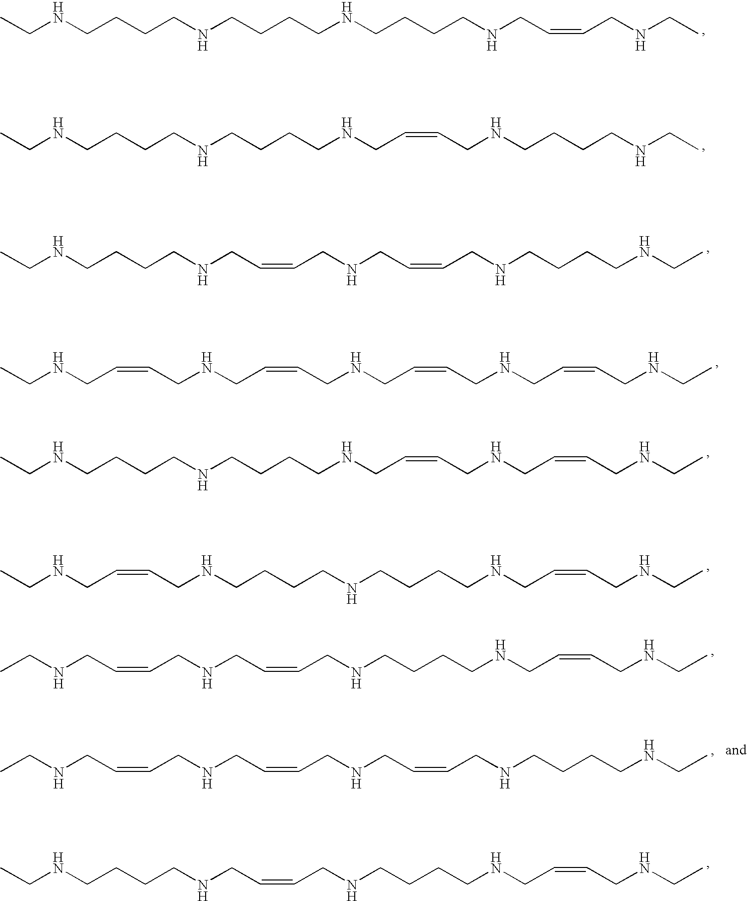
and all salts, hydrates, solvates, and stereoisomers thereof.

In another embodiment, the conformationally restricted polyamine analog is selected from among the group of compounds of the formula:
E-NH—B-A-B—NH—B-A-B—NH—B-A-B—NH(—B-A-B—NH)ₓ-E
wherein A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of a single bond, C₁-C₆ alkyl, and C₂-C₆ alkenyl; E is independently selected from the group consisting of H, C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; and x is an integer from 2 to 16; with the proviso that either at least one A moiety is selected from the group consisting of C₂-C₆ alkenyl, C₂-C₆ alkynyl, C₃-C₆ cycloalkyl, C₃-C₆ cycloaryl, and C₃-C₆ cycloalkenyl, or at least one B moiety is selected from the group consisting of C₂-C₆ alkenyl; and all salts, hydrates, solvates, and stereoisomers thereof. In another embodiment, x is 4, 6, 8, or 10. In another embodiment, x is 4. In another embodiment, x is 6. In another embodiment, x is 8. In another embodiment, x is 10. In one embodiment, the only conformational restriction of the polyamine analog is due to a carbon-carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 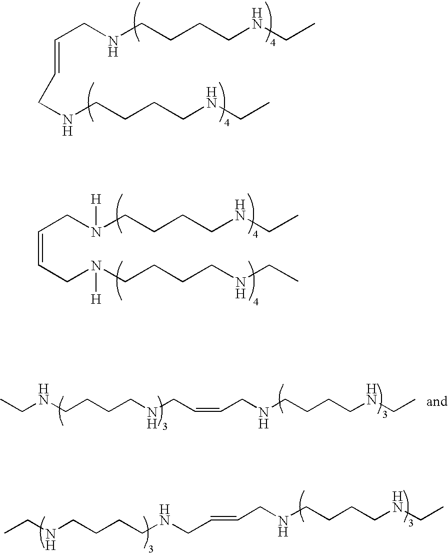
and all salts, hydrates, solvates, and stereoisomers thereof.

In another embodiment, the conformationally restricted polyamine analog is selected from among the group of compounds of the formula
E-NH—B-A-B—NH—B-A-B—NH—B-A-B—NH(—B-A-B—NH)ₓ-E
wherein A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of a single bond, C₁-C₆ alkyl, and C₂-C₆ alkenyl; E is independently selected from the group consisting of C₁-C₆ alkyl, C₁-C₆ alkanol, C₃-C₆ cycloalkanol, and C₃-C₆ hydroxyaryl, with the proviso that at least one E moiety be selected from the group consisting of C₁-C₆ alkanol, C₃-C₆ cycloalkanol, and C₃-C₆ hydroxyaryl; and x is an integer from 0 to 16; and all salts, hydrates, solvates, and stereoisomers thereof. In one embodiment, the only conformational restriction of the polyamine analog is due to a carbon-carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 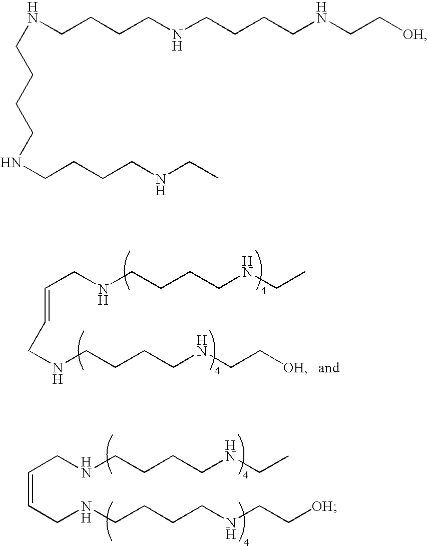
and all salts, hydrates, solvates, and stereoisomers thereof.

In another embodiment, the conformationally restricted polyamine analog is selected from among the group of compounds of the formula
E-NH-D-NH—B-A-B—NH-D-NH-E
wherein A is independently selected from the group consisting of C₂-C₆ alkene and C₃-C₆ cycloalkyl, cycloalkenyl, and cycloaryl; B is independently selected from the group consisting of a single bond and C₁-C₆ alkyl and alkenyl; D is independently selected from the group consisting of C₁-C₆ alkyl and alkenyl, and C₃-C₆ cycloalkyl, cycloalkenyl, and cycloaryl; E is independently selected from the group consisting of H, C₁-C₆ alkyl and alkenyl; and all salts, hydrates, solvates, and stereoisomers thereof. In one embodiment, the only conformational restriction of the polyamine analog is due to a carbon-carbon double bond (an ethenyl group, C═C) in the molecule. In another embodiment, the only conformational restriction of the polyamine analog is due to a cycloalkyl group, such as a cyclopropyl group, in the molecule.

Specific embodiments of compounds of this type include 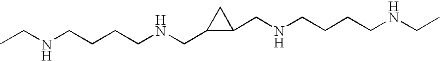
(CGC-11093, formerly SL-11093); 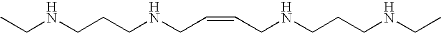
(CGC-11047, formerly SL-11047); and all salts, hydrates, solvates, and stereoisomers thereof.

In another embodiment, the conformationally restricted polyamine analog is selected from macrocyclic polyamines of the formula: 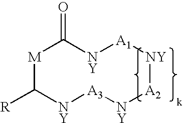
where A₁, each A₂ (if present), and A₃ are independently selected from C₁-C₈ alkyl; where each Y is independently selected from H or C₁-C₄ alkyl; where M is selected from C₁-C₄ alkyl; where k is 0, 1, 2, or 3; and where R is selected from C₁-C₃₂ alkyl; and all salts, hydrates, solvates, and stereoisomers thereof. In additional embodiments, the Y group is —H or —CH₃. In another embodiment, A₁, each A₂ (if present), and A₃ are independently selected from C₂-C₄ alkyl. In yet another embodiment, M is —CH₂—.

In another embodiment, the conformationally restricted polyamine analog is selected from macrocyclic polyamine analogs of the formula 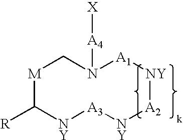
where A₁, each A₂ (if present), and A₃ are independently selected from C₁-C₈ alkyl; where A₄ is selected from C₁-C₈ alkyl or a nonentity; where X is selected from —H, -Z, —CN, —NH₂, —C(═O)—C₁-C₈ alkyl, or —NHZ, with the proviso that when A₄ is a nonentity, X is —H, —C(═O)—C₁-C₈ alkyl, or -Z; where Z is selected from the group consisting of an amino protecting group, an amino capping group, an amino acid, and a peptide; where each Y is independently selected from H or C₁-C₄ alkyl; where M is selected from C₁-C₄ alkyl; where k is 0, 1, 2, or 3; and where R is selected from C₁-C₃₂ alkyl; and all salts, hydrates, solvates, and stereoisomers thereof. In certain embodiments, A₄ is a nonentity. In other embodiments, X is -Z, and -Z is —H. In other embodiments, X is -Z, and -Z is 4-morpholinocarbonyl. In other embodiments, X is -Z and -Z is acetyl. In other embodiments, X is -Z and -Z is t-Boc or Fmoc. In other embodiments, Y is —CH₃. In other embodiments, M is —CH₂—. In still further embodiments, k is 1. In further embodiments, A₁ and A₃ are —CH₂CH₂CH₂—. In still further embodiments, —CH₂CH₂CH₂CH₂—. In still further embodiments, R is —C₁₃H₂₇. In yet further embodiments, one or more of the specific limitations on A₄, X, Z, Y, M, k, A₁, A₃, and R are combined.

In further embodiments of these macrocyclic polyamine analog compounds, A₄ is C₁-C₈ alkyl, X is —NHZ, and Z is selected from one of the 20 genetically encoded amino acids (alanine, cysteine, aspartic acid, glutamic acid, phenylalanine, glycine, histidine, isoleucine, lysine, methionine, asparagine, proline, glutamine, arginine, serine, threonine, valine, tryptophan, tyrosine), a peptide of the formula acetyl-SKLQL-, a peptide of the formula acetyl-SKLQ-β-alanine-, or a peptide of the formula acetyl-SKLQ-. In these cases, where Z is an amino acid or peptide, the therapeutic agent to be used is a polyamine-amino acid conjugate or polyamine-peptide conjugate.

In another embodiment, the conformationally restricted polyamine analog is CGC-11047. In another embodiment, the conformationally restricted polyamine analog is CGC-11093. In another embodiment, the conformationally restricted polyamine analog is CGC-11144. In another embodiment, the the conformationally restricted polyamine analog is CGC-11150.

In another embodiment, the invention embraces a method of treating ocular disease, comprising administering one or more polyamine analogs to a subject with an ocular disease in a therapeutically effective amount. Preferably, the polyamine analog is a conformationally restricted polyamine analog. In one embodiment, the only conformational restriction of the polyamine analog is due to a carbon-carbon double bond (an ethenyl group, C═C) in the molecule. In another embodiment, the only conformational restriction of the polyamine analog is due to a cycloalkyl group, such as a cyclopropyl group, in the molecule. The ocular disease can be macular degeneration. In one embodiment, the ocular disease is dry macular degeneration. In another embodiment, the ocular disease is wet macular degeneration. The method embraces administration of the polyamine analog, which can be a conformationally restricted polyamine analog, in an amount sufficient to reduce retinal neovascularization, to suppress retinal neovascularization, or to delay the development of retinal neovascularization. The invention also embraces administration of the polyamine analog or conformationally restricted polyamine analog in an amount sufficient to reduce choroidal neovascularization, to suppress choroidal neovascularization,or to delay the development of choroidal neovascularization. The invention also embraces administration of the polyamine analog or conformationally restricted polyamine analog in an amount sufficient to cause regression of retinal neovascularization or regression of choroidal neovascularization. The invention also embraces administration of the polyamine analog or conformationally restricted polyamine analog in an amount sufficient to prevent retinal neovascularization or prevent choroidal neovascularization. The reduction, suppression, delay, regression, or prevention of retinal neovascularization or choroidal neovascularization can be either partially or substantially complete. In another embodiment, the conformationally restricted polyamine analog is CGC-11047. In another embodiment, the conformationally restricted polyamine analog is CGC-11093. In another embodiment, the conformationally restricted polyamine analog is CGC-11144. In another embodiment, the the conformationally restricted polyamine analog is CGC-11150.

In another embodiment, the polyamine analog or conformationally restricted polyamine analog is administered as a preventive or prophylactic measure. In one embodiment, the only conformational restriction of the polyamine analog is due to a carbon-carbon double bond (an ethenyl group, C═C) in the molecule. In another embodiment, the only conformational restriction of the polyamine analog is due to a cycloalkyl group, such as a cyclopropyl group, in the molecule. The polyamine analog or conformationally restricted polyamine analog can be administered to patients at risk of retinal neovascularization, choroidal neovascularization or macular degeneration, including wet macular degeneration, at varying intervals and via various methods of administration. Patients at risk of retinal neovascularization, choroidal neovascularization or macular degeneration include, but are not limited to, patients with breaks or tears in Bruch's membrane, dry macular degeneration, extensive drusen deposits, soft drusen deposits, or large confluent drusen deposits. Patients at risk of retinal neovascularization, choroidal neovascularization or macular degeneration also include patients with pigmentary changes in the macula or hypopigmented areas in the macula. Other patients at risk of retinal neovascularization, choroidal neovascularization or macular degeneration include patients with at least one mutation in, or at least one high-risk allele for, a gene involved in development of macular degeneration. Other patients at risk of retinal neovascularization, choroidal neovascularization or macular degeneration also include patients with at least one mutation in, or at least one high-risk allele of, PLEKHA1; patients with at least one mutation in, or at least one high-risk allele of, LOC387715; and/or patients with at least one mutation in, or at least one high-risk allele of, complement factor H (CFH). Other patients at risk of retinal neovascularization, choroidal neovascularization or macular degeneration also include patients with at least one mutation in, or at least one high-risk allele of, genes ABCR, ABCA4, APOE, fibulin 5 (FBLN5), FBLN6 (Hemicentin-1), ELOVL4, TLR4, PRSS11, GRK5, and/or RGS10. Other patients at risk of retinal neovascularization, choroidal neovascularization or macular degeneration also include patients with at least one consanguineous family member affected by retinal neovascularization, choroidal neovascularization or macular degeneration.

In one embodiment, the polyamine analog or conformationally restricted polyamine analog is present in a formulation suitable for ophthalmic administration, where the formulation comprises a polyamine analog or conformationally restricted polyamine analog and a pharmaceutical carrier suitable for ophthalmic administration. In another embodiment, the polyamine analog or conformationally restricted polyamine analog is present in a formulation suitable for periocular administration, where the formulation comprises a polyamine analog or conformationally restricted polyamine analog and a pharmaceutical carrier suitable for periocular administration. In another embodiment, the polyamine analog or conformationally restricted polyamine analog is present in a formulation suitable for periocular injection, where the formulation comprises a polyamine analog or conformationally restricted polyamine analog and a pharmaceutical carrier suitable for periocular injection. The ophthalmic formulations can be administered by topical application to the eye, by injection, or can be surgically implanted in various locations in the eye or tissues associated with the eye, such as intraocular, intravitreal, vitreous chamber, vitreous body, subretinal, periocular, retrobulbar, subconjunctival, or subTenons. In one embodiment, the ophthalmic formulation comprises a polyamine analog or a conformationally restricted polyamine analog and an appropriate buffer system. In another embodiment, the ophthalmic formulation comprises a physiologically balanced irrigating solution. In another embodiment, the ophthalmic formulation comprises Lactated Ringers Solution.

In one embodiment, the invention embraces administration of a polyamine analog or a conformationally restricted polyamine analog about once a week for about two to about twelve months. In another embodiment, the invention embraces administration of a polyamine analog or a conformationally restricted polyamine analog about once every two weeks for about two to about twelve months. In another embodiment, the invention embraces administration of a polyamine analog or a conformationally restricted polyamine analog about once every three weeks for about two to about twelve months. In another embodiment, the aforementioned administration regimens comprise periocular administration of the polyamine analog or conformationally restricted polyamine analog. In another embodiment, the aforementioned administration regimens comprise periocular injection of the polyamine analog or conformationally restricted polyamine analog. In another embodiment, the aforementioned administration regimens comprise administration of CGC-11144. In another embodiment, the aforementioned administration regimens comprise administration of CGC-11150. In another embodiment, the aforementioned administration regimens comprise administration of CGC-11093. In another embodiment, the aforementioned administration regimens comprise administration of CGC-11047.

In another embodiment, the invention embraces administration of a polyamine analog or a conformationally restricted polyamine analog in a sustained release formulation. In one embodiment, the sustained release formulation is implanted into the eye. In another embodiment, the sustained release formulation is implanted into the periocular tissue.

In another embodiment, the invention embraces a polyamine analog or a conformationally restricted polyamine analog in a sustained release formulation, comprising a polyamine analog or a conformationally restricted polyamine analog in a sustained release formulation or sustained release device. In another embodiment, the invention embraces a polyamine analog or a conformationally restricted polyamine analog in a sustained release formulation suitable for administration or implantation in or near the eye, comprising a polyamine analog or a conformationally restricted polyamine analog in a sustained release formulation or sustained release device suitable for administration or implantation in or near the eye. In another embodiment, the invention embraces a polyamine analog or a conformationally restricted polyamine analog in a sustained release formulation suitable for administration or implantation in the periocular tissue, comprising a polyamine analog or a conformationally restricted polyamine analog in a sustained release formulation or sustained release device suitable for administration or implantation in the periocular tissue.

### BRIEF DESCRIPTION OF THE DRAWINGS

FIG. 1 depicts data indicating that systemic administration of the polyamine analogs, CGC-11144 or CGC-11150, causes statistically significant suppression of choroidal neovascularization (CNV). Each panel shows the results of an independent experiment investigating the effect of intraperitoneal (ip) injection of CGC-11144 (A and B) or CGC-11150 (C and D) on the area of CNV at Bruch's membrane rupture sites. Experimental mice were given ip injections twice a week of 10 mg/kg (A and C) or 20 mg/kg (B and D), and compared to control mice with the same genetic background treated twice a week with ip injection of vehicle. Each bar represents the mean (±SEM) area of CNV calculated from the total number of rupture sites for which measurements were taken in each group (n). Injections of 10 (A) or 20 mg/kg (B) of CGC-11144 or injection of 10 (C) or 20 mg/kg (D) of CGC-11150 resulted in small, but significant reductions in CNV. *p<0.05 for difference from vehicle control by unpaired t-test. Each experimental group had its own vehicle control group.

FIG. 2 depicts data indicating that intraocular injection of CGC-11144 or CGC-11150 suppresses choroidal neovascularization (CNV). Eyes that had no treatment after rupture of Bruch's membrane showed large areas of CNV (A and D). Fellow eyes that were injected with vehicle 0 and 7 days after rupture of Bruch's membrane also had large areas of CNV (B and E). Eyes injected with 20 μg of CGC-11144 (C) or CGC-11150 (F) on days 0 and 7 after rupture of Bruch's membrane had areas of CNV that appeared much smaller. Measurement of CNV areas by image analysis showed that eyes injected with CGC-11144 (G) or CGC-11150 (H) had significantly less CNV than corresponding controls (no injection or vehicle-injected fellow eye). The bars show the mean (±SEM) area of CNV calculated from the total number of rupture sites for which measurements were taken in each group (n). †p<0.0001 for difference from no treatment group by linear mixed model with Dunnett's method for multiple comparisons; *p<0.0001 for difference from vehicle control group (fellow eyes) by linear mixed model with Dunnett's method for multiple comparisons.

FIG. 3 depicts data indicating that intravitreous injection of CGC-11144 also causes regression of established choroidal neovascularization (CNV), but alters retinal function and structure. FIG. 3A shows the results of an experiment in which fifteen mice had rupture of Bruch's membrane at 3 locations in each eye; after 7 days, 5 mice were perfused with fluorescein-labeled dextran and the baseline area of CNV was measured. The remaining 10 mice were given an intravitreous injection of 20 μg of CGC-11144 in one eye and vehicle in the fellow eye on days 7 and 10 and then CNV area was measured at each rupture site at 14 days after laser (n=30 in each group). The mean area of CNV was significantly smaller in eyes injected with CGC-11144 compared to vehicle-injected fellow eyes, or compared to the baseline area of CNV measured at 7 days. This indicates that intravitreous injection of CGC-11144 caused regression of established CNV. †p<0.0009 for difference from baseline amount of CNV at 7 days by linear mixed model with Dunnett's method for multiple comparisons. *p<0.0001 for difference from vehicle-injected fellow eye at 14 days by linear mixed model with Dunnett's method. FIG. 3B shows the results of experiments in which mice were given an intravitreous injection of 2, 4, or 20 μg of CGC-11144 in one eye and PBS in the fellow eye. After 3 days, ERGs were performed as described in Methods. Eyes injected with 4 or 20 μg of CGC-11144 had a significant decrease in a-wave amplitudes, and eyes injected with 2, 4, or 20 μg of CGC-11144 had a significant decrease in b-wave amplitudes compared to PBS (p<0.05 by ANOVA). FIG. 3C and FIG. 3D show that, two weeks after intravitreous injection of 20 μg of CGC-11144, there was substantial disruption of the morphology of the retina, particularly the inner retina. In comparison, in FIG. 3E and FIG. 3F, two weeks after intravitreous injection of PBS, the retina had a normal appearance. (In FIGS. 3C-3F, retinal sections were stained with hematoxylin and eosin; FIG. 3C and FIG. 3E, 40×; FIG. 3D and FIG. 3F, 100×.)

FIG. 4 depicts data indicating that periocular injection of CGC-11144 suppresses choroidal neovascularization (CNV), causes regression of established CNV, and has no deleterious effects on retinal function or structure. In FIG. 4A, eyes that had no treatment after rupture of Bruch's membrane showed large areas of CNV 14 days after rupture of Bruch's membrane. In FIG. 4B, eyes given periocular injections of vehicle 3 times a week also had large areas of CNV. In FIG. 4C, eyes given periocular injections of 200 μg of CGC-11144 three times a week appeared to have smaller areas of CNV. FIG. 4D depicts the results of measurement of CNV areas by image analysis showing .that eyes injected with CGC-11144 had significantly less CNV than corresponding controls (no injection or vehicle-injected fellow eye). †p<0.0001 for difference from no treatment by mixed model with Dunnett's method for multiple comparisons; *p<0.0001 for difference from fellow eye by mixed model with Dunnett's method. FIG. 4E shows the results of experiments with fifteen mice that had rupture of Bruch's membrane at 3 locations in each eye. After 7 days, 5 mice were perfused with fluorescein-labeled dextran and the baseline area of CNV was measured. The remaining 10 mice were given periocular injections of 200 μg of CGC-11144 in one eye and vehicle in the fellow eye on days 7, 10, and 13 and then CNV area was measured at each rupture site at 14 days after laser treatment. The mean area of CNV (n=30 rupture sites for each group) was significantly smaller in eyes injected with CGC-11144 compared to vehicle-injected fellow eyes, or compared to the baseline area of CNV measured at 7 days. This indicates that periocular injection of CGC-11144 caused regression of established CNV. †p<0.0001 for difference from baseline amount of CNV at 7 days by linear mixed model and Dunnett's method *p<0.0001 for difference from vehicle-injected fellow eye at 14 days by linear mixed model and Dunnett's method. FIG. 4F shows results for mice (n=20) that received daily periocular injection of 200 μg of CGC-11144 in one eye and vehicle in the fellow eye for 14 days; ERGs were then performed on each eye. There was no difference in mean a-wave or b-wave amplitudes between eyes injected with CGC-11144 or vehicle controls. FIG. 4G presents data that eyes treated with daily periocular injections of 200 μg of CGC-11144 for 14 days showed normal-appearing retinas that looked identical to those in eyes treated with vehicle (shown in FIG. 4H).

FIG. 5 shows data indicating that periocular injections with the polyamine synthesis inhibitor D,L-α-difluoromethyl-omithine (DFMO) cause regression of choroidal neovascularization (CNV), but do not enhance the regression caused by CGC-11144. Thirty-four adult C57BL/6 mice had rupture of Bruch's membrane in 3 locations in each eye and after 7 days, 8 mice were perfused with fluorescein-labeled dextran and the baseline area of CNV was measured. The remaining mice were given daily periocular injections of 200 μg of CGC-11144, 100 μg of DFMO, or both in one eye and vehicle in the fellow eye; CNV area was measured at 14 days after laser in these mice. The cross (†) applies to all 3 treatment groups and indicates that the area of CNV for each is significantly less (p<0.01) than the day 7 baseline level of CNV by linear mixed model with Dunnett's method for multiple comparisons. The asterisk (*) applies to all 3 treatment groups and indicates that the area of CNV for each is significantly less (p<0.01) than the area of CNV in vehicle control fellow eyes by linear mixed model and Dunnett's method. Combined treatment with DFMO and CGC-11144 was not significantly different from treatment with CGC-11144 alone.

FIG. 6 shows data indicating that periocular injections of CGC-11144 cause apoptosis in CNV lesions. Adult female C57BL/6 mice had laser-induced rupture of Bruch's membrane in 3 locations in one eye. On days 7 and 8 after laser treatment, mice received a periocular injection of 200 μg of CGC-11144 or vehicle. On day 9 after laser, the mice were euthanized, eyes were frozen in OCT, and 10 μm serial sections were cut through CNV lesions. A representative section from a mouse treated with CGC-11144 shows several red TUNEL-stained nuclei located within the CNV lesion (FIG. 6A, arrows), which is delineated by Griffonia simplicifolia staining in an adjacent section (FIG. 6B). In contrast, no TUNEL-positive cells are seen within a CNV lesion (arrows) in an eye that received periocular injections of vehicle (FIG. 6C and FIG. 6D).

FIG. 7 depicts the effect of a single periocular injection of CGC-11144, CGC-11047 and CGC-11093 on suppression of CNV.

FIG. 8 depicts the effect of a single periocular injection of CGC-11144, CGC11047 and CGC-11093 on regression of CNV.

FIG. 9 depicts the duration of anti-angiogenic activity after a single periocular injection. Panel A shows the effects of CGC-11144, CGC-11047 and CGC-11093 injected one week prior to laser-induced rupture. Panel B shows the effects of CGC-11144, CGC-11047 and CGC-11093 injected two weeks prior to laser-induced rupture. Panel C shows the effects of CGC-11047 and CGC-11093 injected three weeks prior to laser-induced rupture.

FIG. 10 depicts the effect of periocular injections of CGC-11144, CGC11047 and CGC-11093 on oxygen-induced ischemic retinal neovascularization. Panels A and C depicts suppression of retinal neovascularization and Panel B depicts the regression of established retinal neovascularization.

FIG. 11 depicts the effect of periocular injections of CGC-11144, CGC11047 and CGC-11093 on retinal neovascularization in rhodopsin/VEGF transgenic mice.

### DETAILED DESCRIPTION OF THE INVENTION

The term “ocular” means “of, relating to, or connected with the eye.”

A “subject” or a “patient” refers to a vertebrate, preferably a mammal, more preferably a human. The polyamine analogs described herein or incorporated by reference herein are used to treat vertebrates, preferably mammals, more preferably humans.

“Treating” or “to treat” a disease using the methods of the invention is defined as administering one or more polyamine analogs, with or without additional therapeutic agents, in order to palliate, ameliorate, stabilize, reverse, slow, delay, prevent, reduce, or eliminate either the disease or the symptoms of the disease, or to retard or stop the progression of the disease or of symptoms of the disease. “Therapeutic use” of the polyamine analogs is defined as using one or more polyamine analogs to treat a disease, as defined above. A “therapeutically effective amount” is an amount sufficient to treat a disease, as defined above. Prevention or suppression can be partial or total.

By “undesirable cell proliferation” is meant any condition where cells are growing or multiplying, and such growth or multiplication is undesirable (for example, causing disease or unwanted symptoms). In one embodiment, benign tumors are excluded from conditions characterized by undesirable cell proliferation. In another embodiment, malignant (cancerous) tumors are excluded from conditions characterized by undesirable cell proliferation. In another embodiment, both benign and malignant (cancerous) tumors are excluded from conditions characterized by undesirable cell proliferation.

By “neovascularization” is meant the formation of new blood vessels, such as capillaries. While neovascularization can be a desirable effect, such as in wound healing or embryonic development, it can be an undesirable effect in several diseases, such as in macular degeneration, where neovascularization and/or subsequent leakage from the neovasculature leads to impairment of retinal function.

By “polyamine analog” is meant an organic cation structurally similar but non-identical to naturally occurring polyamines such as spermine and/or spermidine and their precursor, diamine putrescine. By a “polyamine”, a term well-understood in the art, is meant any of a group of aliphatic, straight-chain amines derived biosynthetically from amino acids; polyamines are reviewed in Marton et al. (1995) Ann. Rev. Pharm. Toxicol. 35: 55-91. Polyamine analogs can be branched or un-branched. Polyamine analogs include, but are not limited to, BE-4444 [1,19-bis (ethylamino)-5,10,15-triazanonadecane]; BE-333 [N1,N11]-diethylnorspermine; DENSPM; 1,11-bis(ethylamino)-4,8-diazaundecane; thermine; Warner-Parke-Davis]; BE-33 [N1,N7-bis(ethyl)norspermidine]; BE-34 [N1,N8-bis(ethyl)spermidine]; BE-44 [N1,N9-bis(ethyl)homopermidine]; BE-343 [N1,N12-bis(ethyl)spermine; diethylspermine-N1-N12; DESPM]; BE-373 [N,N′-bis(3-ethylamino)propyl)-1,7-heptane diamine, Merrell-Dow]; BE-444 [N1,N14-bis(ethyl)homospermine; diethylhomospermine-N1-N14]; BE-3443 [1,17-bis(ethylamino)-4,9,14-triazaheptadecane]; and BE-4334 [1,17-bis(ethylamino)-5,9,13-triazaheptadecane]; 1,12-Me₂-SPM [1,12-dimethylspermine]. See also Feuerstein et al. (1991); Gosule et al. (1978) J. Mol. Biol. 121: 311-326; Behe et al. (1981) Proc. Natl. Acad. Sci. USA 78: 1619-23; Jain et al. (1989) Biochem. 28: 2360-2364; Basu et al. (1990) Biochem. J. 269: 329-334; Porter et al. (1988), Advances in Enzyme Regulation, Pergamon Press, pp. 57-79; Frydman et al. (1992) Proc. Natl. Acad. Sci. USA 89: 9186-9191; and Fernandez et al. (1994) Cell Mol. Biol. 40: 933-944.

By “conformationally restricted” is meant that, in a polyamine analog, at least two amino groups in the molecule are locked or limited in spatial configuration relative to each other. The amino groups within the molecule may be primary, secondary, tertiary, or quartenary, and are preferably primary or secondary amino groups, more preferably secondary amino groups. The relative movement of two amino groups can be restricted, for example, by incorporation of a cyclic or unsaturated moiety between them (exemplified, but not limited to, a ring, such as a three-carbon ring, four-carbon ring, five-carbon-ring, six-carbon ring, or a double or triple bond, such as a double or triple carbon bond). Polyamines can also be constrained by incorporation of two or more amino groups into a macrocyclic structure. Groups restricting conformational flexibility by means of steric hindrance, yet favorable to the therapeutic effects of the compound, can also be used. A conformationally restricted polyamine analog can comprise at least two amino groups which are conformationally restricted relative to each other; a polyamine analog can also further comprise amino groups which are not conformationally restricted relative to other amino groups. Flexible molecules such as spermine and BE-444 can have a myriad of conformations and are therefore not conformationally restricted. Conformationally restricted polyamine analogs include, but are not limited to, the compounds disclosed in International Patent Application WO 98/17624, U.S. Pat. No. 5,889,061, and U.S. Pat. No. 6,392,098; the compounds disclosed in WO 00/66587 and U.S. Pat. No. 6,794,545; and the compounds disclosed in U.S. Patent Application Publication Nos. 2003/0072715, 2003/0195377, and International Patent Applications WO 02/10142, and WO 03/050072. Several of these compounds are depicted below in Table 1. All of the polyamine analog compounds (both conformationally restricted polyamine analog compounds and non-conformationally restricted polyamine analog compounds) disclosed in those patents or patent applications, including but not limited to the specification, claims, tables, examples, figures, and schemes of those patents or patent applications, are expressly incorporated by reference herein as compounds useful in the invention. The conformationally restricted polyamine analog compounds disclosed in those patents or patent applications, including but not limited to the specification, claims, tables, examples, figures, and schemes of those patents or patent applications, are expressly incorporated by reference herein as preferred compounds useful in the invention.

In certain embodiments, the saturated oligoamines disclosed in U.S. Patent Application Publication No. 2003/0130356 can be used for treatment of ocular diseases, and all oligoamine compounds disclosed therein, including but not limited to the specification, claims, tables, examples, figures, and schemes of that patent application, are expressly incorporated by reference herein as compounds useful in the invention.

In certain additional embodiments, the polyamine analog-peptide conjugates disclosed in U.S. Pat. No. 6,649,587 can be used for treatment of ocular diseases, and all polyamine analog-peptide conjugates disclosed therein, including but not limited to the specification, claims, tables, examples, figures, and schemes of that patent, are expressly incorporated by reference herein as compounds useful in the invention.

In certain additional embodiments, the polyamine analog-amino acid conjugates disclosed in International Patent Application WO 02/38105 can be used for treatment of ocular diseases, and all polyamine analog-amino acid conjugates disclosed therein, including but not limited to the specification, claims, tables, examples, figures, and schemes of that patent application, are expressly incorporated by reference herein as compounds useful in the invention.

One preferred subset of polyamine analogs and conformationally restricted polyamine analogs are those containing 8, 10, 12, or 14 nitrogen atoms. Such compounds include CGC-11144 and CGC-11150 (also known as SL-11144 and SL-11150, respectively), each of which contains 10 nitrogens.

Another preferred subset of polyamine analogs and conformationally restricted analogs comprises the compounds known as CGC-11093 and CGC-11047 (also known as SL-11093 and SL-11047, respectively), each of which contains 4 nitrogens.

The invention includes the use of all of the compounds described herein or incorporated by reference herein, including any and all stereoisomers, salts, hydrates and solvates of the compounds described herein or incorporated by reference herein. The invention also includes the use of all compounds described herein or incorporated by reference herein in their non-salt, non-hydrate/non-solvate form. Particularly preferred are pharmaceutically acceptable salts. Pharmaceutically acceptable salts are those salts which retain the biological activity of the free bases and which are not biologically or otherwise undesirable. The desired salt may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of the compounds with amino acids, such as aspartate salts and glutamate salts, can also be prepared.

The invention also includes all stereoisomers of the compounds, including diastereomers and enantiomers, as well as mixtures of stereoisomers, including, but not limited to, racemic mixtures. Unless stereochemistry is explicitly indicated in a structure, the structure is intended to embrace all possible stereoisomers of the compound depicted.

The term “alkyl” refers to saturated aliphatic groups including straight-chain, branched-chain, cyclic groups, and combinations thereof, having the number of carbon atoms specified, or if no number is specified, having up to 12 carbon atoms, with preferred subsets of alkyl groups including C₁-C₁₂, C₁-C₁₀, C₁-C₈, C₁-C₆, and C₁-C₄ alkyl groups. “Straight-chain alkyl” or “linear alkyl” groups refers to alkyl groups that are neither cyclic nor branched, commonly designated as “n-alkyl” groups. Examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cyclic groups can consist of one ring, including, but not limited to, groups such as cycloheptyl, or multiple fused rings, including, but not limited to, groups such as adamantyl or norbornyl.

“Substituted alkyl” refers to alkyl groups substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂—CF₃, and other perfluoro and perhalo groups.

“Hydroxyalkyl” specifically refers to alkyl groups having the number of carbon atoms specified substituted with one —OH group. Thus, “C₃ linear hydroxyalkyl” refers to —CH₂CH₂CHOH—, —CH₂CHOHCH₂—, and —CHOHCH₂CH₂—.

The term “alkenyl” refers to unsaturated aliphatic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃; and —CH₂—CH₂-cyclohexenyl, where the ethyl group can be attached to the cyclohexenyl moiety at any available carbon valence. The term “alkynyl” refers to unsaturated aliphatic groups including straight-chain (linear), branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Aryl” or “Ar” refers to an aromatic carbocyclic group having a single ring (including, but not limited to, groups such as phenyl) or multiple condensed rings (including, but not limited to, groups such as naphthyl or anthryl), and includes both unsubstituted and substituted aryl groups. “Substituted aryls” refers to aryls substituted with one or more substituents, including, but not limited to, groups such as alkyl, alkenyl, alkynyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, 2-ethylthiophenyl, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, thiophene, or furyl) or multiple condensed rings (including, but not limited to, examples such as imidazolyl, indolizinyl or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as alkyl, alkenyl, alkynyl, benzyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) as well as the carbon atoms of the group can be substituted. The heteroatom(s) can also be in oxidized form, if chemically possible.

The term “alkylaryl” refers to an alkyl group having the number of carbon atoms designated, appended to one, two, or three aryl groups.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, and t-butoxy.

The term “alkanoate” as used herein refers to an ionized carboxylic acid group, such as acetate (CH₃C(═O)—O⁽⁻¹⁾), propionate (CH₃CH₂C(═O)—O⁽⁻¹⁾), and the like. “Alkyl alkanoate” refers to a carboxylic acid esterified with an alkoxy group, such as ethyl acetate (CH₃C(═O)—O—CH₂CH₃). “co-haloalkyl alkanoate” refers to an alkyl alkanoate bearing a halogen atom on the alkanoate carbon atom furthest from the carboxyl group; thus, ethyl co-bromo propionate refers to ethyl 3-bromopropionate, methyl ω-chloro n-butanoate refers to methyl 4-chloro n-butanoate, etc.

The terms “halo” and “halogen” as used herein refer to Cl, Br, F or I substituents.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 2nd Ed. (John Wiley & Sons, Inc., New York). Amino protecting groups include, but are not limited to, mesitylenesulfonyl (Mes), benzyloxycarbonyl (CBz or Z), t-butyloxycarbonyl (Boc), t-butyldimethylsilyl (TBDIMS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Hydroxyl protecting groups include, but are not limited to, Fmoc, TBDIMS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4-nitrophenethyloxymethyloxycarbonyl).

Examples of compounds useful in the invention are depicted in Table 1. While some of the compounds are depicted as salts, such as the hydrochloride salt, it is to be understood that the disclosure in the table embraces all salts, hydrates, and solvates of the compounds depicted therein, as well as the non-salt, non-hydrate/non-solvate form of the compound, as is well understood by the skilled artisan. Table 1 includes both non-conformationally restricted polyamine analogs and conformationally restricted polyamine analogs. While both types of polyamine analogs are useful in the invention, the conformationally restricted polyamine analogs are preferred for use in the invention.

TABLE 1

| Compound | Structure |
|---|---|
| CGC-11027 (formerly SL-11027) | 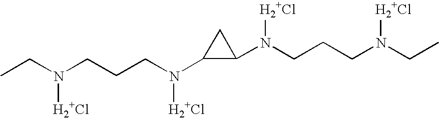 |
| CGC-11028 (formerly SL-11028) | 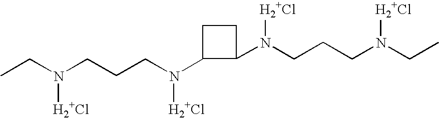 |
| CGC-11029 (formerly SL-11029) | 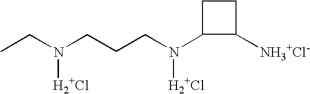 |
| CGC-11033 (formerly SL-11033) | 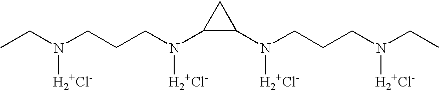 |
| CGC-11034 (formerly SL-11034) | 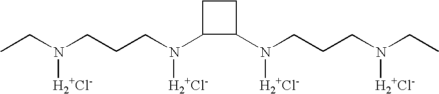 |
| CGC-11035 (formerly SL-11035) | 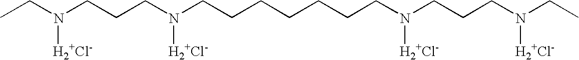 |
| CGC-11036 (formerly SL-11036) | 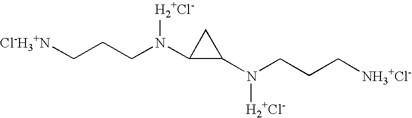 |
| CGC-11037 (formerly SL-11037) | 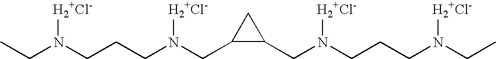 |
| CGC-11038 (formerly SL-11038) | 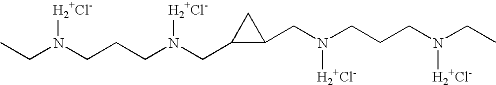 |
| CGC-11043 (formerly SL-11043) | 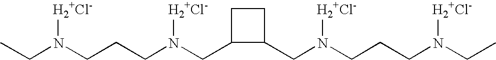 |
| CGC-11044 (formerly SL-11044) | 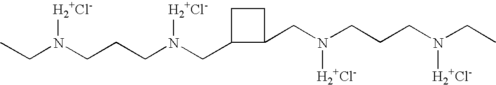 |
| CGC-11047 (formerly SL-11047) | 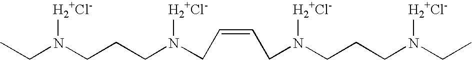 |
| CGC-11048 (formerly SL-11048) | 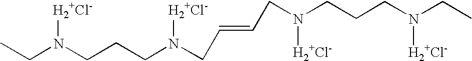 |
| CGC-11050 | BnNH(CH₂)₄NHBn |
| (formerly | ·2HCl |
| SL-11050) | |
| CGC-11061 | EtNH(CH₂)₄NH(CH₂)₄NH(CH₂)₄NH(CH₂)₄—NHEt |
| (formerly | ·5HCl |
| SL-11061) | |
| CGC-11093 (formerly SL-11093) | 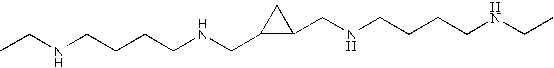 |
| CGC-11094 (formerly SL-11094) | 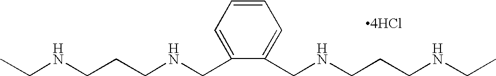 |
| CGC-11098 (formerly SL-11098) | 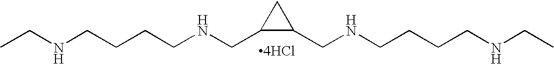 |
| CGC-11099 (formerly SL-11099) | 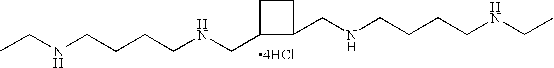 |
| CGC-11100 (formerly SL-11100) | 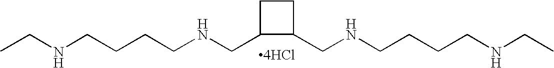 |
| CGC-11101 (formerly SL-11101) | 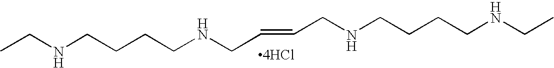 |
| CGC-11102 (formerly SL-11102) | 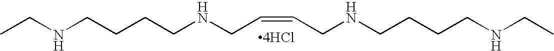 |
| CGC-11103 (formerly SL-11103) | 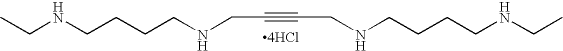 |
| CGC-11104 (formerly SL-11104) | 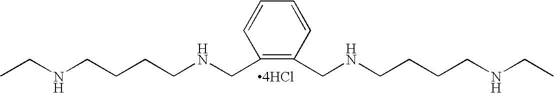 |
| CGC-11105 (formerly SL-11105) | 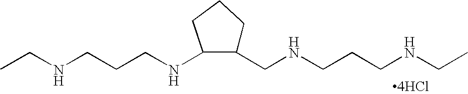 |
| CGC-11108 (formerly SL-11108) | 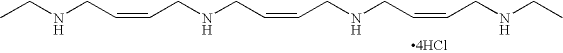 |
| CGC-11114 (formerly SL-11114) | 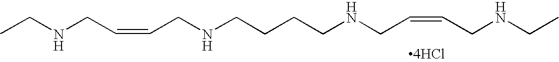 |
| CGC-11119 (formerly SL-11119) | 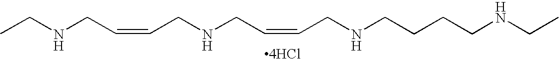 |
| CGC-11090 (formerly SL-11090) | 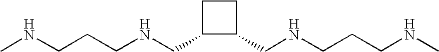 |
| CGC-11091 (formerly SL-11091) | 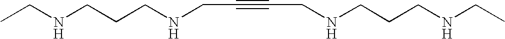 |
| CGC-11092 (formerly SL-11092) | 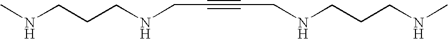 |
| CGC-11101 (formerly SL-11101) | 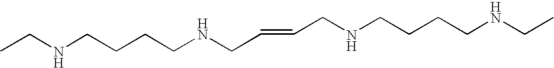 |
| CGC-11103 (formerly SL-11103) | 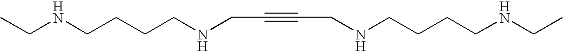 |
| CGC-11114 (formerly SL-11114) | 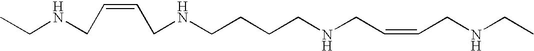 |
| CGC-11118 (formerly SL-11118) | 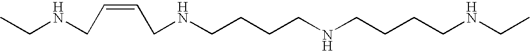 |
| CGC-11121 (formerly SL-11121) | 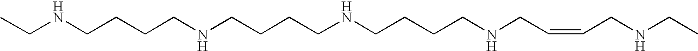 |
| CGC-11122 (formerly SL-11122) | 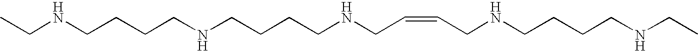 |
| CGC-11123 (formerly SL-11123) | 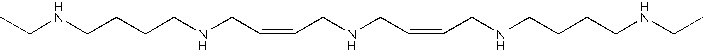 |
| CGC-11124 (formerly SL-11124) | 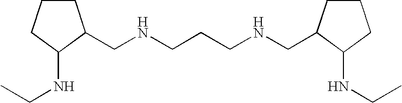 |
| CGC-11126 (formerly SL-11126) | 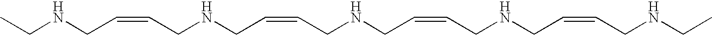 |
| CGC-11127 (formerly SL-11127) | 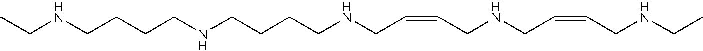 |
| CGC-11128 (formerly SL-11128) | 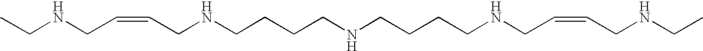 |
| CGC-11129 (formerly SL-11129) | 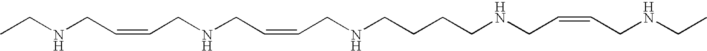 |
| CGC-11130 (formerly SL-11130) | 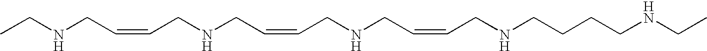 |
| CGC-11132 (formerly SL-11132) | 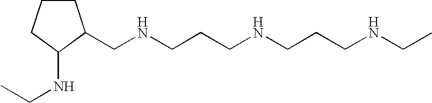 |
| CGC-11133 (formerly SL-11133) | 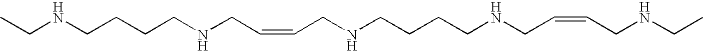 |
| CGC-11134 (formerly SL-11134) | 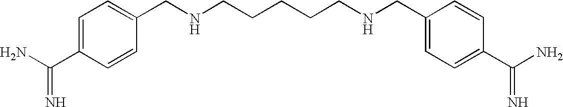 |
| CGC-11135 (formerly SL-11135) | 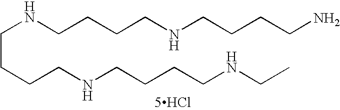 |
| CGC-11136 (formerly SL-11136) | 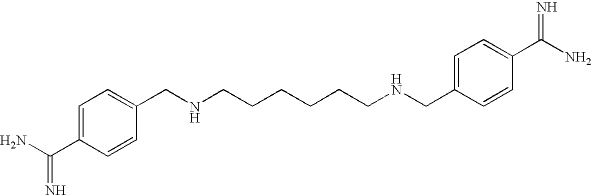 |
| CGC-11137 (formerly SL-11137) | 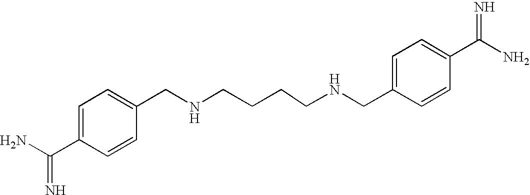 |
| CGC-11141 (formerly SL-11141) | 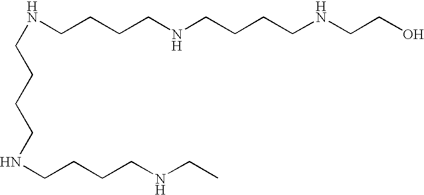 |
| CGC-11143 (formerly SL-11143) | 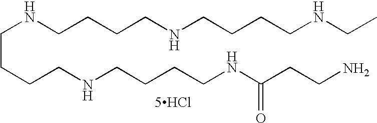 |
| CGC-11144 (formerly SL-11144) | 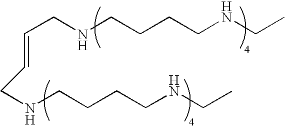 |
| CGC-11150 (formerly SL-11150) | 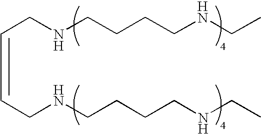 |
| CGC-11155 (formerly SL-11155) | 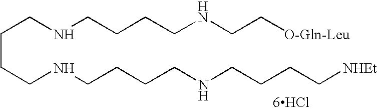 |
| CGC-11157 (formerly SL-11157) | 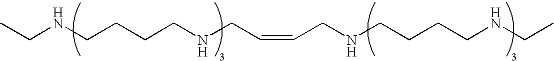 |
| CGC-11158 (formerly SL-11158) | 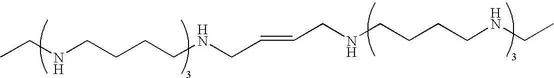 |
| CGC-11201 (formerly SL-11201) | 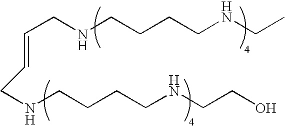 |
| CGC-11202 (formerly SL-11202) | 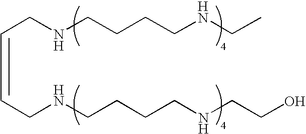 |
| CGC-11174 (formerly SL-11174) | 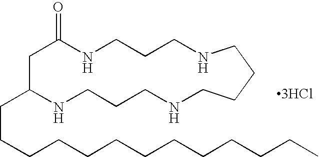 |
| CGC-11197 (formerly SL-11197) | 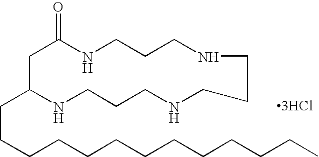 |
| CGC-11199 (formerly SL-11199) | 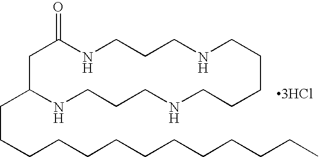 |
| CGC-11200 (formerly SL-11200) | 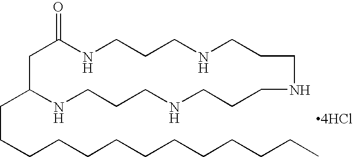 |
| CGC-11208 (formerly SL-11208) | 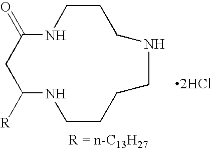 |
| CGC-11238 (formerly SL-11238) | 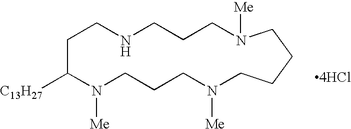 |
| CGC-11239 (formerly SL-11239) | 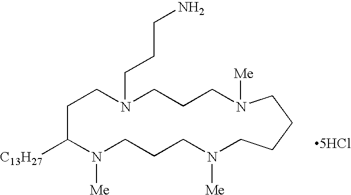 |

Ocular Diseases

The invention embraces methods of treating a variety of ocular diseases.

These diseases include diseases characterized by neovascularization or other undesired growth in regions of the eye, including, but not limited to, neovascularization of the retina, neovascularization of the cornea (such as that caused by trachoma, infections, inflammation, transplantations or trauma), proliferative vitreoretinopathy, diabetic retinopathy, diabetic retinal edema, diabetic macular edema, ischemic retinopathy, hypertensive retinopathy, occlusive retinopathy, retinal vascular diseases, branch or central vein occlusion, neovascularization due to retinal arterial occlusion, ocular ischemic syndrome or carotid artery disease, vasculitis, cystoid macular edema, parafoveal retinal telangiectasis or arterial macroaneurysms, radiation retinopathy, sickle cell retinopathy, peripheral retinal neovascularization such as Coats disease (retinal telangiectasis), retinopathy of prematurity, neovascularization subsequent to trauma, neovascularization subsequent to infection, neovascularization subsequent to transplantation, neovascularization subsequent to retinal detachment or retinal degeneration, neovascularization involved in glaucoma (such as anterior chamber and/or anterior chamber angle neovascularization), choroidal neovascularization (CNV), and subretinal neovascularization. Other diseases which can result in neovascularization include laceration of the eye or puncture of the eye, intraocular foreign bodies, and exposure to laser light or other radiation which damages the eye. One particular disease which can be treated by the methods of the invention is macular degeneration, including age-related macular degeneration. Both “dry” macular degeneration and “wet” macular degeneration can be treated by the methods of the invention.

Dry macular degeneration occurs when drusen deposits interfere with the normal functioning of Bruch's membrane and the retinal pigment epithelium. The retinal cells overlying the drusen deposits atrophy, with resulting loss of vision. Drusen deposits may coalesce into larger drusen plaques, causing atrophy of large areas of the retina; this process is known as “geographic atrophy.” Patients with drusen deposits, particularly “soft” drusen deposits (soft drusen deposits are drusen deposits with ill-defined margins, as compared to hard drusen deposits which have sharp, well-defined borders), are also at risk of developing wet macular degeneration. Other risk factors include pigmentary changes in the macula or hypopigmented areas in the macula.

Wet macular degeneration occurs when neovascularization occurs behind the macula. The neovasculature is excessively permeable, and leakage and bleeding from the neovascular tissue causes severe damage to the macula.

Experiments have demonstrated that polyamines can suppress or prevent neovascularization after laser-induced injury to Bruch's membrane (see the Examples, below; in the Examples, “suppression” is used synonymously with “prevention”). As breaks often occur in aged Bruch's membrane, when a subject or patient presents with breaks in Bruch's membrane, the polyamines can be used prophylatically in order to suppress or prevent macular degeneration in that subject or patient. The polyamines can also be used prophylatically in patients displaying other characteristics which are associated with risk of developing macular degeneration, such as extensive drusen deposits, large confluent drusen deposits, or soft drusen deposits. Given the increased risk of development of wet neovascularization in patients presenting with dry macular degeneration, dry macular degeneration can be treated with compounds of the invention in order to suppress or prevent neovascularization and wet macular degeneration in light of the suppressive and preventive properties of the compounds.

Other risk factors which have been identified for macular degeneration include particular allele variants of three genes, PLEKHA1, LOC387715 and complement factor H (CFH). In particular, two single nucleotide polymorphisms in these genes are implicated: Ala69Ser in LOC387715, and Tyr402His in CFH. See Jakobsdottir et al., Am. J. Hum. Genet. 77: 389-407 (2005); Rivera et al. Hum. Mol. Genet., electronic publication ahead of print on Sep. 20, 2005; doi: 10.1093/hmg/ddi353 at World-Wide Web address hmg.oxfordjoumals(.org). The data of Rivera et al. show a disease odds ratio of 57.6 in individuals homozygous for risk alleles at both CFH and LOC387715, as compared with the baseline non-risk genotype. Individuals heterozygous or homozygous for one or both of these higher-risk alleles, and in particular individuals homozygous for one or both higher-risk alleles, would be good candidates for prophylactic therapy with compounds of the invention. Other individuals at higher risk for macular degeneration include individuals with mutations or higher-risk alleles in ABCR e.g. ABCA₄ (Allikmets et al., Science 277: 1805-1807 (1997)), APOE (Klaver et al., Am. J. Hum. Genet. 63: 200-206 (1998)), fibulin 5 (FBLN5) (Stone et al., N. Engl. J. Med., 351: 346-353 (2004)), FBLN6 (Hemicentin-1) (Schultz et al., Hum. Mol Genet. 12: 3315-3323 (2003)), ELOVL4 (Conley et al., Hum. Mol. Genet. 14: 1991-2002 (2005)), and TLR4 (Zareparsi et al., Hum. Mol. Genet. 14: 1449-1455 (2005)).

Modes of Administration

Compounds useful in the methods of the invention can be administered to a patient or subject (preferably a human patient or subject) via any route known in the art, including, but not limited to, those disclosed herein. Methods of administration include, but are not limited to, systemic, transpleural, intravenous, oral, intraarterial, intramuscular, topical, via inhalation (e.g. as mists or sprays), via nasal mucosa, subcutaneous, transdermal, intraperitoneal, gastrointestinal, and directly to the eye or tissues surrounding the eye. The compounds described or incorporated by reference for use herein can be administered in the form of tablets, pills, powder mixtures, capsules, granules, injectables, creams, solutions, suppositories, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

A preferred route of administration is to the eye or the tissues associated with the eye. The compounds can be administered topically to the eye, as in eye drops or eye washes. The compounds can also be administered via injection to the eye (intraocular injection) or to the tissues associated with the eye. The compounds can be administered via subconjunctival injection, trans-septal injection, intravitreal injection, transpleural injection, subretinal injection, periocular injection, sub-Tenon's injection, or retrobulbar injection. The compounds can also be administered to the subject or patient as an implant. Preferred implants are biocompatible and/or biodegradable sustained release formulations which gradually release the compounds over a period of time. Ocular implants for drug delivery are well-known in the art; see, e.g., U.S. Pat. Nos. 5,501,856, 5,476,511, and 6,331,313. The compounds can also be administered to the subject or patient using iontophoresis, including, but not limited to, the iontophoretic methods described in U.S. Pat. No. 4,454,151 and U.S. Patent Application Publication Nos. 2003/0181531 and 2004/0058313.

The pharmaceutical dosage form which contains the compounds for use in the invention is conveniently admixed with a non-toxic pharmaceutical organic carrier or a non-toxic pharmaceutical inorganic carrier. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the compound(s) to retain its pharmacological activity in the body. Formulations for parenteral and nonparenteral drug delivery are known in the art and are set forth in Remington: The Science and Practice of Pharmacy, 20th Edition, Lippincott, Williams & Wilkins (2000). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; desiccants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulfate. The tablets can be coated according to methods well known in standard pharmaceutical practice. Liquid forms for ingestion can be formulated using known liquid carriers, including aqueous and non-aqueous carriers such as sterile water, sterile saline, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, the compounds for use in the invention can be administered as injectable dosages of a solution or suspension of the compound in a physiologically acceptable diluent or sterile liquid carrier such as water, saline, or oil, with or without additional surfactants or adjuvants. An illustrative list of carrier oils would include animal and vegetable oils (e.g., peanut oil, soy bean oil), petroleum-derived oils (e.g., mineral oil), and synthetic oils.

For injectable unit doses, sterile liquids such as water, saline, aqueous dextrose and related sugar solutions are preferred liquid carriers. For administration to the eye, the polyamine analog(s) is formulated as a composition suitable for ophthalmic administration according to methods known in the art.

The compounds of the present invention can be administered as solutions, suspensions, or emulsions (dispersions) in a suitable ophthalmic formulation. An appropriate buffer system (e.g., sodium phosphate, sodium acetate, sodium citrate, or sodium borate) may be added. Physiologically balanced irrigating solutions as part of the ophthalmic formulations for the compounds may be used when the compositions are administered intraocularly or periocularly. As used herein, the term “physiologically balanced irrigating solution” means a solution which is adapted to maintain the physical structure and function of tissues during invasive or noninvasive medical procedures. This type of solution will typically contain electrolytes, such as sodium, potassium, calcium, magnesium and/or chloride; an energy source, such as dextrose; and a buffer to maintain the pH of the solution at or near physiological levels. Various solutions of this type are known (e.g., Lactated Ringers Solution). BSS Registered TM Sterile Irrigating Solution and BSS Plus® Sterile Intraocular Irrigating Solution (Alcon Laboratories, Inc., Fort Worth, Tex., USA) are examples of physiologically balanced intraocular irrigating solutions.

The compounds of the present invention can be administered as topical ophthalmic formulations and can include ophthalmologically acceptable preservatives, surfactants, viscosity enhancers, buffers, sodium chloride and water to form aqueous sterile ophthalmic solutions and suspensions. Sterile ophthalmic gel formulations can be prepared by suspending a compound in a hydrophilic base prepared from a combination of, for example, Carbopol® 940 (a carboxyvinyl polymer available from the B.F. Goodrich Company) according to published formulations for analogous ophthalmic preparations. Preservatives and tonicity agents may also be incorporated in such gel formulations.

The pharmaceutical unit dosage chosen is preferably fabricated and administered to provide a defined final concentration of drug either in the blood, or in tissues of the eye and/or tissues associated with the eye. The optimal effective concentration of the compounds of the invention can be determined empirically and will depend on the type and severity of the disease, route of administration, disease progression and health, mass and body area of the patient. Such determinations are within the skill of one in the art. Examples of dosages which can be used for systemic administration include, but are not limited to, an effective amount within the dosage range of about 0.1 μg/kg to about 300 mg/kg, or within about 1.0 μg/kg to about 40 mg/kg body weight, or within about 10 μg/kg to about 20 mg/kg body weight, or within about 0.1 mg/kg to about 20 mg/kg body weight, or within about 1 mg/kg to about 20 mg/kg body weight, or within about 0.1 mg/kg to about 10 mg/kg body weight, or within about within about 1 mg/kg to about 10 mg/kg body weight, or within about 0.1 μg/kg to about 10 mg/kg body weight. Examples of dosages which can be used for systemic administration when based on body surface area (expressed in square meters, or m²) include, but are not limited to, an effective amount within the dosage range of about 0.1 μg/m² to about 300 mg/m² body surface area, or within about 10 μg/m² to about 300 mg/m² body surface area, or within about 100 μg/m² to about 300 mg/m² body surface area, or within about 1 mg/m² to about 300 mg/m² body surface area, or within about 10 mg/m² to about 300 mg/m² body surface area, or within about 10 mg/m² to about 200 mg/m² body surface area, or within about 10 mg/m² to about 120 mg/m² body surface area, or within about 40 mg/m² to about 120 mg/m² body surface area, or within about 60 mg/m² to about 100 mg/m² body surface area. For intraocular and intravitreous administration or injection, examples of dosages which can be used include, but are not limited to, about any of 1 μg, 5 μg, 10 μg, 15 μg, 20 μg, 25 μg, 30 μg, 50 μg, 75 μg, 100 μg, 200 μg, 300 μg, 400 μg, 500 μg, 600 μg, 700 μg, 800 μg, 900 μg, 1 mg, 2 mg, 3 mg, 4 mg, or 5 mg per eye. For periocular administration or injection, examples of dosages which can be used include, but are not limited to, about any of 25 μg, 50 μg, 100 μg, 150 μg, 200 μg, 250 μg, 300 μg, 350 μg, 400 μg, 500 μg, 600 μg, 700 μg, 750 μg, 800 μg, 900 μg, 1 mg, 1.5 mg, 2 mg, 2.5 mg, 3 mg, 3.5 mg, 4 mg, 4.5 mg, 5 mg, 6 mg, 7 mg, 8 mg, 9 mg, 10 mg, 12.5 mg, 15 mg, 20 mg, 25 mg, 30 mg, 40 mg, or 50 mg per eye. The dosages may be administered in a single daily dose, or the total daily dosage may be administered in divided dosage of two, three or four times daily. Dosages may also be administered less frequently than daily, for example, six times a week, five times a week, four times a week, three times a week, twice a week, about once a week, about once every two weeks, about once every three weeks, about once every four weeks, about once every six weeks, about once every two months, about once every three months, about once every four months, or about once every six months.

In one embodiment, the invention embraces administration of a polyamine analog or a conformationally restricted polyamine analog about once a week for about two to about twelve months. In another embodiment, the invention embraces administration of a polyamine analog or a conformationally restricted polyamine analog about once every two weeks for about two to about twelve months. In another embodiment, the invention embraces administration of a polyamine analog or a conformationally restricted polyamine analog about once every three weeks for about two to about twelve months. In another embodiment, the invention embraces administration of a polyamine analog or a conformationally restricted polyamine analog about once a month for about two to about twelve months. In another embodiment, the invention embraces administration of a polyamine analog or a conformationally restricted polyamine analog about once every two months for about two to about twelve months. In another embodiment, the invention embraces administration of a polyamine analog or a conformationally restricted polyamine analog about once every three months for about three to about twelve months. In another embodiment, the invention embraces administration of a polyamine analog or a conformationally restricted polyamine analog about once every four months for about four to about twelve months. In another embodiment, the invention embraces administration of a polyamine analog or a conformationally restricted polyamine analog about once every five months for about five to about fifteen months. In another embodiment, the invention embraces administration of a polyamine analog or a conformationally restricted polyamine analog about once every six months for about six to about twelve months. In another embodiment, the invention embraces administration of a polyamine analog or a conformationally restricted polyamine analog about once a week, about once every two weeks, about once every three weeks, about once a month, about once every two months, about once every three months, about once every four months, about once every five months, about or once every six months, for an indefinite period of time, or until particular clinical endpoints are met. In another embodiment, the aforementioned administration regimens comprise periocular administration of the polyamine analog or conformationally restricted polyamine analog. In another embodiment, the aforementioned administration regimens comprise periocular injection of the polyamine analog or conformationally restricted polyamine analog. In another embodiment, the aforementioned administration regimens comprise administration of CGC-11047. In another embodiment, the aforementioned administration regimens comprise administration of CGC-11093. In another embodiment, the aforementioned administration regimens comprise administration of CGC-11144. In another embodiment, the aforementioned administration regimens comprise administration of CGC-11150.

In one embodiment, the invention embraces ophthalmic formulations of the compounds disclosed herein as useful in treating undesired cell proliferation or neovascularization, and in particular for treating macular degeneration, especially wet macular degeneration. The ophthalmic formulations can be administered by topical application to the eye, by injection, or can be surgically implanted in various locations in the eye or tissues associated with the eye, such as intraocular, intravitreal, vitreous chamber, vitreous body, subretinal, periocular, retrobulbar, subconjunctival, or subTenons. In one embodiment, the ophthalmic formulation comprises a polyamine analog or a conformationally restricted polyamine analog and an appropriate buffer system. In another embodiment, the ophthalmic formulation comprises a physiologically balanced irrigating solution. In another embodiment, the ophthalmic formulation comprises Lactated Ringers Solution.

In one embodiment of the invention, the dosages may be administered in a sustained release formulation or a sustained release implant, such as in an implant which gradually releases the compounds for use in the invention over a period of time, and which allow for the drug to be administered less frequently, such as about once a month, about once every 2-6 months, about once every year, or even a single administration which need not be repeated. The sustained release implants, devices or formulations (such as pellets, microspheres, and the like) can be administered by topical application to the eye, by injection, or can be surgically implanted in various locations in the eye or tissues associated with the eye, such as intraocular, intravitreal, vitreous chamber, vitreous body, subretinal, periocular, retrobulbar, subconjunctival, or subTenons. The sustained release formulation may be combined with iontophoretic methods.

The compounds for use in the invention can be administered as the sole active ingredient, or can be administered in combination with another active ingredient. In one embodiment, D,L-α-difluoromethyl-omithine is used as another active ingredient. In another embodiment, L-α-difluoromethyl-omithine is used as another active ingredient. In another embodiment, D-α-difluoromethyl-ornithine is used as another active ingredient.

Kits

The invention also provides articles of manufacture and kits containing materials useful for treating ocular diseases. The article of manufacture comprises a container with a label. Suitable containers include, for example, bottles, vials, and test tubes. The containers may be formed from a variety of materials such as glass or plastic. The container holds a composition having an active agent which is effective for treating the ocular disease. The active agent in the composition is one or more conformationally restricted polyamine analogs, preferably one or more of the conformationally restricted polyamine analogs disclosed herein or incorporated by reference herein. The label on the container indicates that the composition is used for treating ocular diseases such as macular degeneration, and may also indicate directions for use. The container can be a container adapted for administration of the composition to the eye, such as a bottle for eyedrops. The container can also be a container or a unit adapted for implantation or injection into the eye or the tissues surrounding the eye, such as the periocular tissue. The composition present in the container can comprise any of the ophthalmic formulations or compositions described herein.

The invention also provides kits comprising any one or more of a conformationally restricted polyamine analog. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein (methods for treating ocular diseases, such as macular degeneration). The composition present in the kit can comprise any of the ophthalmic formulations or compositions described herein.

In other aspects, the kits may be used for any of the methods described herein, including, for example, to treat a patient or subject suffering from an ocular disease. In some embodiments, the ocular disease is macular degeneration. The kits may include instructions for practicing any of the methods described herein.

The following examples are provided to illustrate various embodiments of the invention, and are not intended to limit the invention in any manner.

### EXAMPLES

The decamines used in certain of the Examples below, CGC-11144 and CGC-11150 (formerly SL-11144 and SL-11150), were synthesized as previously described (U.S. Pat. No. 6,794,545; Valasinas et al., Bioorg. Med. Chem. 11: 4121-4131 (2003); Bacchi et al., Antimicrob. Agents Chemother. 46: 55-61 (2002)). The agents were diluted in phosphate-buffered saline (PBS) for injections. D,L-α-difluoromethyl-omithine (DFMO) was obtained from Sigma (St. Louis, Mo.).

### Example 1

### Mouse Model of Choroidal Neovascularization (CNV): Laser-Induced CNV

Mice were treated in accordance with the recommendations of the Association for Research in Vision and Ophthalmology and the U.S. National Institutes of Health Guide for the Care and Use of Laboratory Animals. Laser photocoagulation-induced rupture of Bruch's membrane was used to generate CNV (see Tobe T. et al., Am J Pathol 153: 1641-1646 (1998)). Briefly, 6 to 8 week old female C57BL/6J mice were anesthetized with ketamine hydrochloride (100 mg/kg body weight) and the pupils were dilated with 1% tropicamide (Alcon Labs, Inc., Forth Worth, Tex.). Three burns of 532 nm diode laser photocoagulation (75 μm spot size, 0.1 seconds duration, 120 mW) were delivered to each retina using the slit lamp delivery system of an OcuLight GL Photocoagulator (Iridex, Mountain View, Calif.) and a hand held cover slide as a contact lens. Bums were performed in the 9, 12, and 3 o'clock positions of the posterior pole of the retina. Production of a bubble at the time of laser, which indicates rupture of Bruch's membrane, is an important factor in obtaining CNV (Tobe T. et al., Am J Pathol 153: 1641-1646 (1998)), so only burns in which a bubble was produced were included.

Two weeks after rupture of Bruch's membrane, mice were anesthetized and perfused with fluorescein-labeled dextran (2×10⁶ average mw, Sigma, St. Louis, Mo.) and choroidal flat mounts were prepared as previously described (Nambu et al., Invest. Ophthalmol. Vis. Sci. 44: 3650-3655 (2003)). Briefly, the eyes were removed, fixed for 1 hour in 10% phosphate-buffered formalin, and the comea and lens were removed. The entire retina was carefully dissected from the eyecup, radial cuts were made from the edge of the eyecup to the equator in all 4 quadrants, and it was flat-mounted in Aquamount. Flat-mounts were examined by fluorescence microscopy using an Axioskop microscope (Zeiss, Thornwood, N.Y.) and images were digitized using a 3 CCD color video camera (IK-TU40A, Toshiba, Tokyo, Japan) and a frame grabber. Image-Pro Plus software (Media Cybernetics, Silver Spring, Md.) was used to measure the area of each CNV lesion. Statistical comparisons were made using a linear mixed model (Verbeke and Molenberghs, Linear Mixed Models for Longitudinal Data. New York, Springer-Verlag, Inc., 2000, pp 93-120). This model is analogous to analysis of variance (ANOVA), but allows analysis of all CNV area measurements from each mouse rather than average CNV area per mouse by accounting for correlation between measurements from the same mouse. The advantage of this model over ANOVA is that it accounts for differing precision in mouse-specific average measurements arising from a varying number of observations among mice. P-values for comparison of treatments were adjusted for multiple comparisons using Dunnett's method.

### Example 2

### Intraperitoneal (ip) Administration of CGC-11144 and CGC-11150

Four independent experiments were done to investigate the effect of intraperitoneal (ip) injections: (1) 8 mice received twice a week ip injections of 10 mg/kg of CGC-11144 and 8 mice received twice a week ip injections of vehicle, (2) 10 mice received three times a week ip injections of 20 mg/kg of CGC-11144 and 8 mice received three times a week ip injections of vehicle, (3) 8 mice received twice a week ip injections of 10 mg/kg of CGC-11150 and 8 mice received twice a week ip injections of vehicle, and (4) 10 mice received three times a week ip injections of 20 mg/kg of CGC-11150 and 9 mice received three times a week ip injections of vehicle.

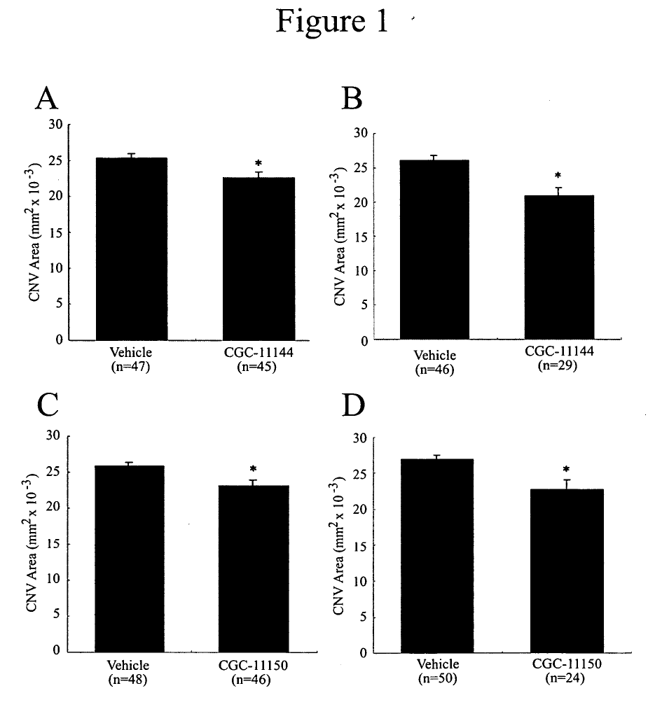

Intraperitoneal (ip) injections of 10 mg/kg of CGC-11144 (FIG. 1A) or CGC-11150 (FIG. 1C) twice a week were well tolerated and caused statistically significant reductions in the size of CNV lesions. Injections of 20 mg/kg of CGC-11144 (FIG. 1B) or CGC-11150 (FIG. 1D) ip twice a week were not well tolerated and several mice died during the 2-week treatment period. Mice that survived showed small statistically significant reductions in CNV lesion size compared to vehicle-injected controls; the reductions were comparable to the reductions seen with the 10 mg/kg doses.

### Example 3

### Intravitreous Administration of CGC-11144 and CGC-11150

Two independent experiments were done to investigate the effect of intravitreous injections: (1) 10 mice received an injection of 20 μg of CGC-11144 in one eye and vehicle in the fellow eye on days 0 and 7 after rupture of Bruch's membrane, (2) 10 mice received an injection of 20 μg of CGC-11150 in one eye and vehicle in the fellow eye on days 0 and 7 after rupture of Bruch's membrane.

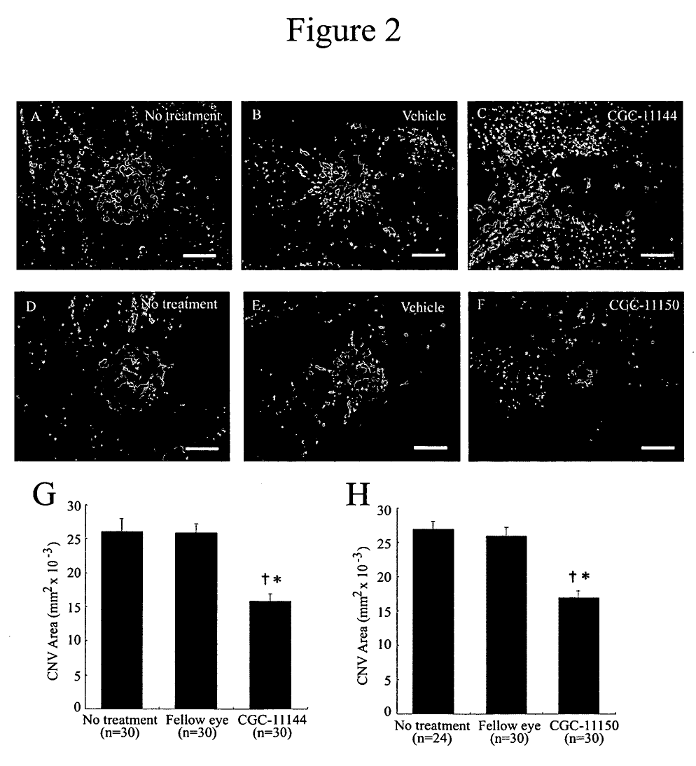

Intravitreous injections of polyamine analogs cause substantial reduction of CNV at Bruch's membrane rupture sites. Eyes that received no treatment had consistent amounts of CNV 14 days after rupture of Bruch's membrane (FIG. 2A and FIG. 2D). Other mice received intravitreous injection of 20 μg of CGC-11144 or CGC-11150 immediately after laser-induced rupture of Bruch's membrane and 7 days after laser in one eye, and vehicle injections in the fellow eye. In eyes that received intravitreous injections of vehicle, the area of CNV at Bruch's membrane rupture sites (FIG. 2B and FIG. 2E) looked very similar to that seen in untreated eyes (FIG. 2A and FIG. 2D). In contrast, the size of CNV lesions at rupture sites appeared smaller in eyes treated with CGC-11144 (FIG. 2C) or CGC-11150 (FIG. 2F). Measurement of CNV area by image analysis showed that eyes injected with CGC-11144 (FIG. 2G) or CGC-11150 (FIG. 2H) had a statistically significant decrease in area of CNV by approximately 40% compared to vehicle-treated eyes or untreated eyes. The lack of a difference between untreated eyes and fellow eyes treated with vehicle suggests that there was no systemic effect from intraocular injection of CGC-11144 or CGC-11150.

### Example 4

### Effect of Intravitreous Injection of CGC-11144 on Retinal Function as Assessed by Electroretinograms (ERG)

Adult female C57BL/6 mice were given an intravitreous injection of 2, 4, or 20 μg of CGC-11144 in one eye and an injection of vehicle in the fellow eye and after 3 days ERGs were recorded as previously described (Okoye et al., J. Neuosci. 23: 4164-4172 (2003)). ERGs were also performed after daily periocular injections of 0.2 mg of CGC-11144 for 2 weeks. Mice were dark-adapted for a standardized 12-hour period overnight and ERG recordings were performed using the Espion ERG Diagnosys (Diagnosys LLL, Littleton, Mass.). All manipulations were done with dim red light illumination. Beginning the same time each morning, mice were anesthetized by ip injection of 25 μl/g body weight of Avertin (Aldrich, Milwaukee, Wis.) diluted 1:39 in PBS. Corneas were anesthetized with a drop of 0.5% proparacaine hydrochloride (Alcon Labs) and pupils were dilated with 1% tropicamide. Mice were placed on a pad heated to 39° C. and platinum electrodes were placed on each cornea after application of gonioscopic prism solution (Alcon Labs). The reference electrode was placed subcutaneously in the anterior scalp between the eyes, and the ground electrode was inserted into the tail. Electrode impedance was balanced for each eye pair measured. The head of the mouse was placed in a standardized position in a Ganzfeld bowl illuminator that assured equal illumination of the eyes. Simultaneous recordings from both eyes were made for eleven intensity levels of white light ranging from −3.00 to +1.40 log cd-s/m². The Espion ERG machine measures the ERG response six times at each flash-intensity and records the average value.

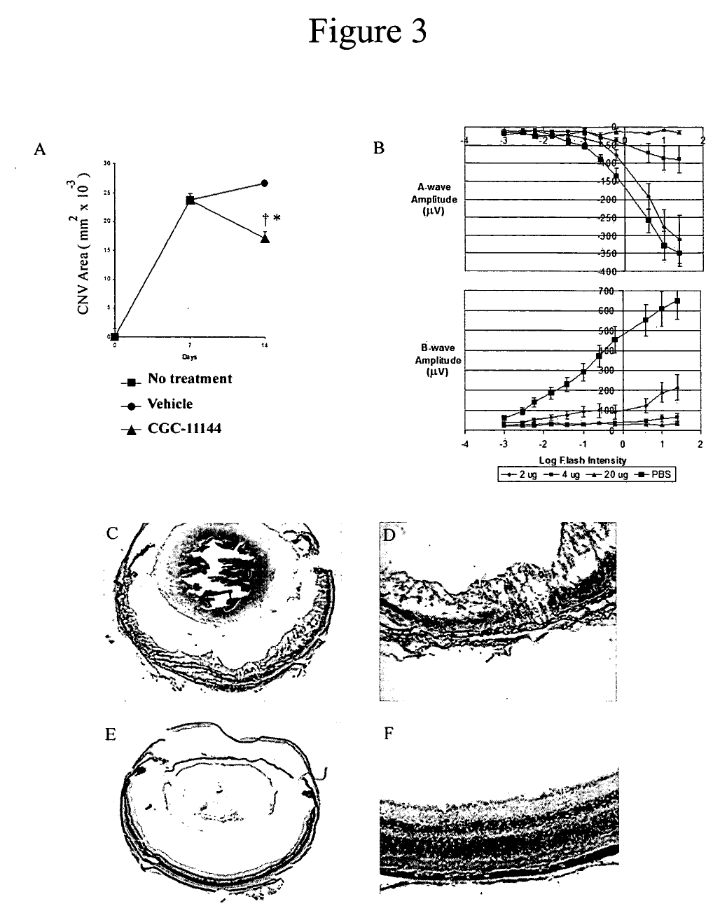

ERG provides an assessment of retinal functioning. Three days after intravitreous injection of 20 μg of CGC-11144 both ERG a- and b-wave amplitudes were dramatically reduced (FIG. 3B). Injection of 10 μg of CGC-11144 also caused a striking decrease in ERG amplitudes, and while a-wave amplitudes were only mildly decreased at highest flash intensities after injection of 2 μg of CGC-11144, b-wave amplitudes were profoundly reduced at all flash intensities. Retinal sections from eyes injected with 20 μg of CGC-11144 illustrate that retinal structure, as well as function, was markedly disrupted (FIGS. 3C and D), compared to fellow eyes injected with vehicle (FIGS. 3E and F). An intravitreous injection of 20 μg of CGC-11150 also causes retinal damage (not shown).

### Example 5

### Periocular Administration of CGC-11144 and CGC-11150

Experiments were also done to investigate the effect of periocular injection of CGC-11144 in which 10 mice received periocular injection three times a week of 0.2 mg of CGC-11144 in 5 μl of PBS in one eye and 5 μl of PBS in the fellow eye. To control for effects from systemic absorption in the fellow eye, 5 mice received no treatment.

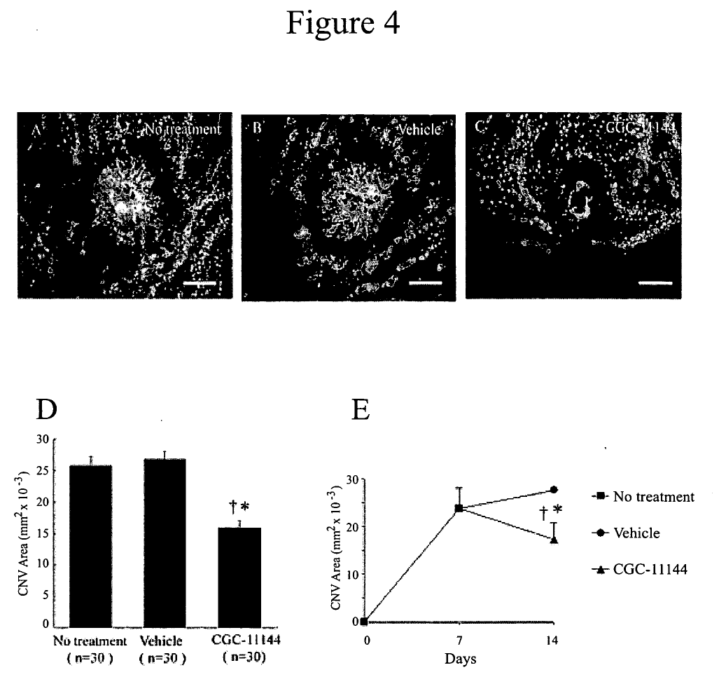

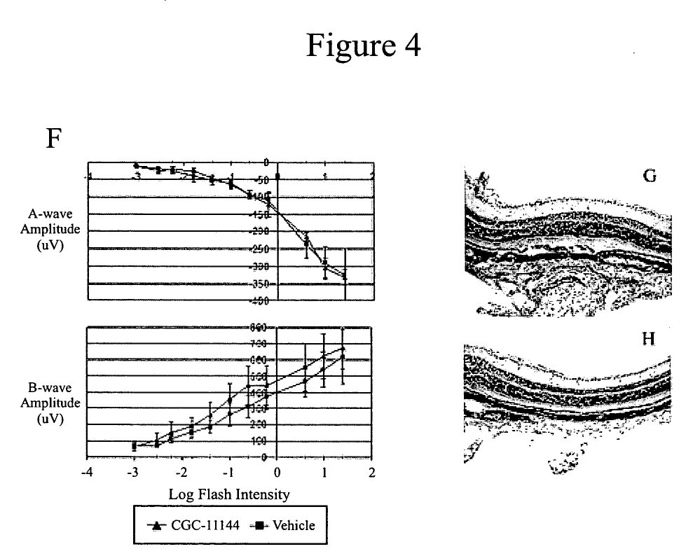

Results of these experiments showed that periocular injection of CGC-11144 can suppress, and cause regression of, CNV with no decrease in retinal function. Fourteen days after rupture of Bruch's membrane there were large areas of CNV in eyes that received no treatment (FIG. 4A) or periocular injections of vehicle (FIG. 4B). The CNV at Bruch's membrane rupture sites appeared smaller in eyes treated 3 times a week with periocular injections of 200 μg of CGC-11144 (FIG. 4C). Measurement of CNV areas by image analysis showed that eyes treated with periocular CGC-11144 had significantly less CNV, with a 40% decrease in area compared to vehicle-treated eyes (FIG. 4D), very similar to the decrease seen from intraocular injection of 20 μg of CGC-11144.

### Example 6

Treatment of established CNV and effect of periocular injection of CGC-11144 on retinal function as assessed by electroretinograms (ERG)

Adult female C57BL/6 mice had laser treatment to three locations in each eye as described above. Only burns in which a bubble was produced were included. After one week, some mice were used to measure the baseline amount of CNV present at 7 days; these mice were perfused 7 days after rupture of Bruch's membrane and the area of CNV at rupture sites was measured on choroidal flat mounts (“7 day baseline eyes”). Other mice were treated with 20 μg of CGC-11144 in one eye and vehicle in the fellow eye on days 7 and 10, or they were treated with periocular injections of 200 μg of CGC-11144 in one eye and vehicle in the fellow eye on days 7, 10, and 13. On day 14, the mice were perfused with fluorescein-labeled dextran and CNV area was measured at Bruch's membrane rupture sites on choroidal flat mounts. In some experiments, mice had rupture of Bruch's membrane and then between days 7 and 14 were given daily periocular injections of 5 μl vehicle in one eye and in the fellow eye received 5 μl containing 100 μg of DFMO, 200 μg of CGC-11144, or a combination of 100 μg of DFMO and 200 μg of CGC-11144.

Intravitreous administration of CGC-11144: FIG. 3 depicts the results of the experiments in which the mice received intravitreous injections of 20 μg of CGC-11144 in one eye and vehicle in the other eye on days 7 and 10. At 14 days after rupture of Bruch's membrane, eyes that had been injected with CGC-11144 had CNV lesions that were significantly smaller in area than those present in fellow eyes that had been treated with vehicle, and also significantly smaller than the area of CNV lesions in the 7 day baseline eyes, indicating that there had been regression of CNV (FIG. 3A).

Periocular administration of CGC-11144: Starting 7 days after rupture of Bruch's membrane, a time point when CNV is well-established, 3 periocular injection of 200 μg of CGC-11144 between days 7 and 14 resulted in a significant decrease in area of CNV (FIG. 4E). Therefore, like intraocular injections of CGC-11144, periocular injections are able to suppress the development of CNV when started at the time of rupture of Bruch's membrane, and cause partial regression of CNV when administered to eyes with established CNV. However, unlike eyes given an intraocular injection of 2 μg or more of CGC-11144, eyes treated with daily periocular injections of 200 μg of CGC-11144 for 14 days had normal ERG a- and b-wave amplitudes that were no different from those in eyes treated with vehicle (FIG. 4F). They also had normal-appearing retinas (FIG. 4G) that were similar in appearance to those from eyes given periocular injections of vehicle (FIG. 4H).

### Example 7

### Periocular Injection of CGC-11144 Results in Apoptosis Within CNV Lesions

On days 7 and 8 after laser-induced rupture of Bruch's membrane, adult female C57BL/6 mice received a periocular injection of 0.2 mg of CGC-11144 or vehicle and then they were euthanized. Eyes were embedded in OCT and 10 μm serial sections were cut through CNV lesions and fixed with 1% paraformaldehyde for 10 minutes at room temperature. TUNEL staining was done using an ApopTag Fluorescein Red Kit (Intergen, Purchase, N.Y.) following the manufacturer's instructions. Adjacent sections were stained for GSA to visualize vascular cells on some sections; TUNEL staining was done to identify cell undergoing apoptosis on adjacent sections.

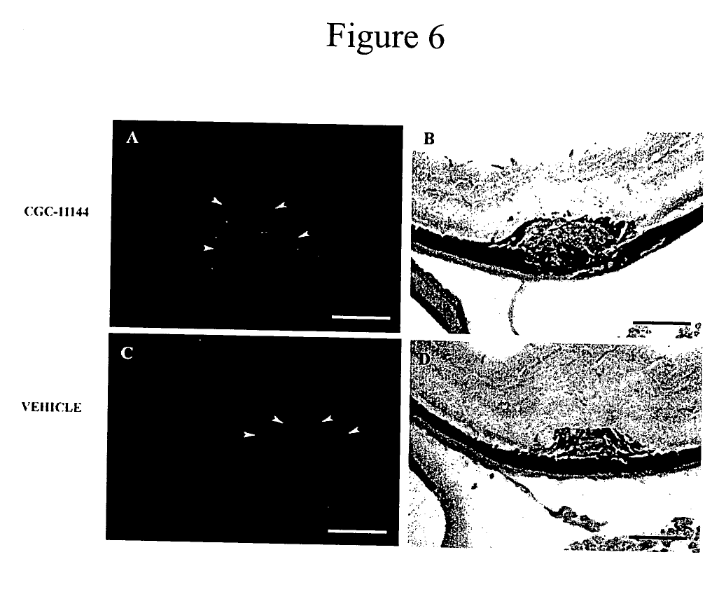

Eyes that had received periocular injections of CGC-11144 showed many TUNEL-stained cells within CNV lesions (FIG. 6A and FIG. 6B), while there was no detectable apoptosis in CNV lesions from eyes injected with vehicle (FIG. 6C and FIG. 6D).

### Example 8

### Daily Periocular Injections of CGC-11144; Periocular Injections of CGC-11144 Combined With DFMO

Daily administration of CGC-11144 was studied to determine whether more frequent administration would increase the inhibitory effect of polyamine analogs on CNV. CGC-11144 was also administered in combination with D,L-α-difluoromethyl-omithine (DFMO) as another potential strategy to increase the inhibitory effect on CNV. Like polyamine analogs, DFMO reduces intracellular polyamine levels, but acts by blocking polyamine synthesis.

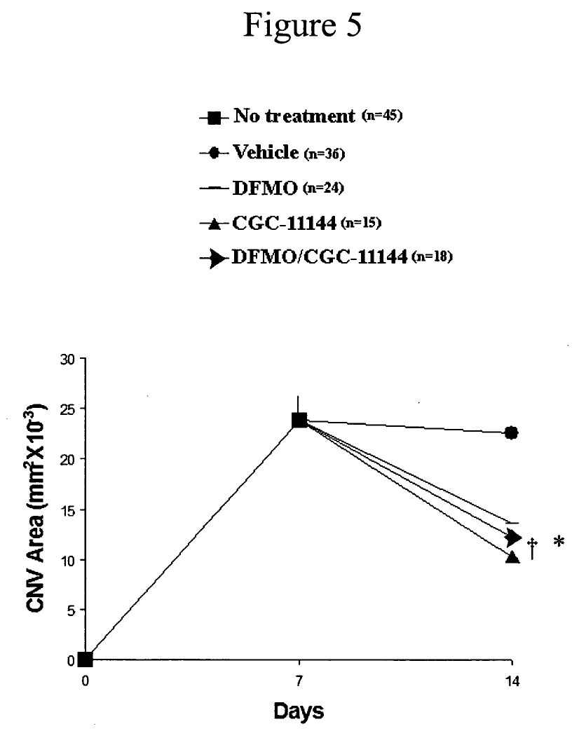

Daily periocular injections of 5 μl containing 100 μl of DFMO between days 7 and 14 resulted in significant reduction in the area of CNV at Bruch's membrane rupture sites compared to the baseline amount present at day 7 (FIG. 5).

Daily periocular injections of 5 μl containing 200 μg of CGC-11144 between days 7 and 14 resulted in significant reduction in CNV area (FIG. 5), very similar to that seen with daily injections of 100 μg of DFMO.

Co-injection of 200 μg of CGC-11144 and 100 μg of DMFO (FIG. 5) did not result in a further enhancement of the regressive effect of either alone.

### Example 9

### Studies on Suppression of CNV Using a Single Periocular Injection

Previous studies demonstrated that multiple periocular injections of CGC-11144 were able to suppress the development of CNV at rupture sites in Bruch's membrane. A study was done to determine the effect of a single periocular injection of the same dose of CGC-11144 given at the time of rupture of Bruch's membrane on the amount of CNV detected two weeks later. In addition to CGC-11144, two other polyamine analogs, CGC-11047 and CGC-11093 were also tested in the single periocular injection studies.

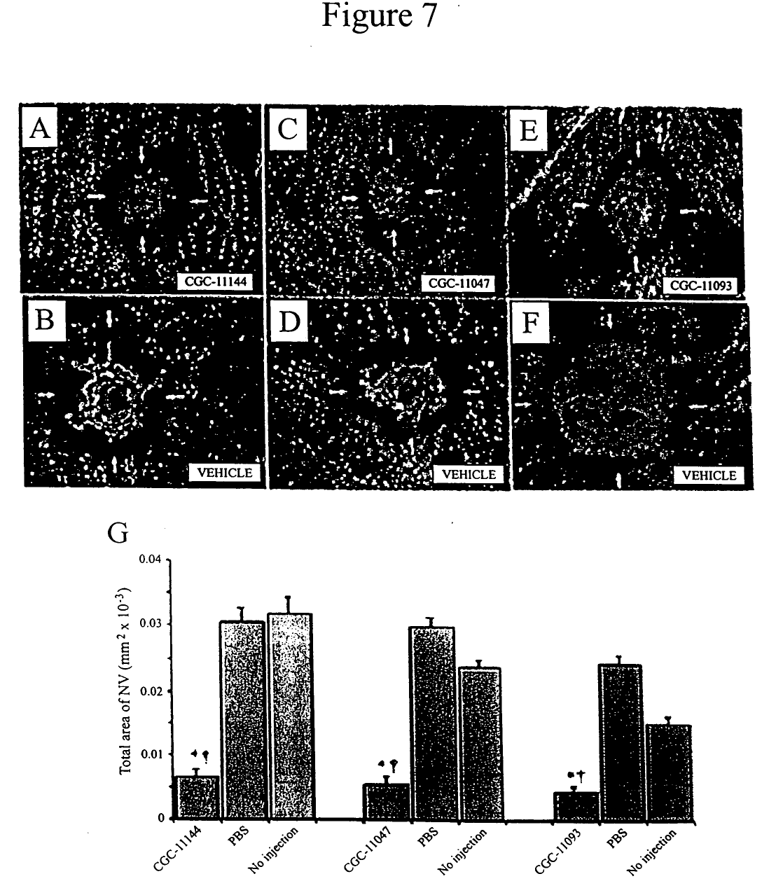

Adult female C57BL/6 mice had laser-induced rupture of Bruch's membrane at 3 locations in each eye as described above in Example 1. Mice received a single periocular injection of 200 μg of CGC-11144, 2 mg of CGC-11047, 1.5 mg of CGC-11093 or vehicle immediately after laser treatment. Two weeks after rupture of Bruch's membrane, mice were perfused with fluorescein-labeled dextran and choroidal flat mounts were prepared. A single periocular injection of CGC-11144 (FIG. 7A), CGC-11047 (FIG. 7C), or CGC-11093 (FIG. 7E) at the time of rupture of Bruch's membrane significantly reduced the amount of CNV when measured on day 14, as compared to vehicle (FIG. 7B, FIG. 7D, and FIG. 7F, respectively). This is illustrated graphically in FIG. 7G.

### Example 10

### Studies on Regression of CNV Using a Single Periocular Injection

To determine the effect of a single periocular injection on established CNV, Bruch's membrane was ruptured by laser photocoagulation in 3 locations in each eye. At day 7 one group of mice was perfused with fluorescein-labeled dextran and the amount of CNV was measured to establish baseline levels. The remaining mice were given a single periocular injection of 200 μg of CGC-11144, 2 mg of CGC-11047, 1.5 mg of CGC-11093 or vehicle and on day 14 the amount of CNV was measured.

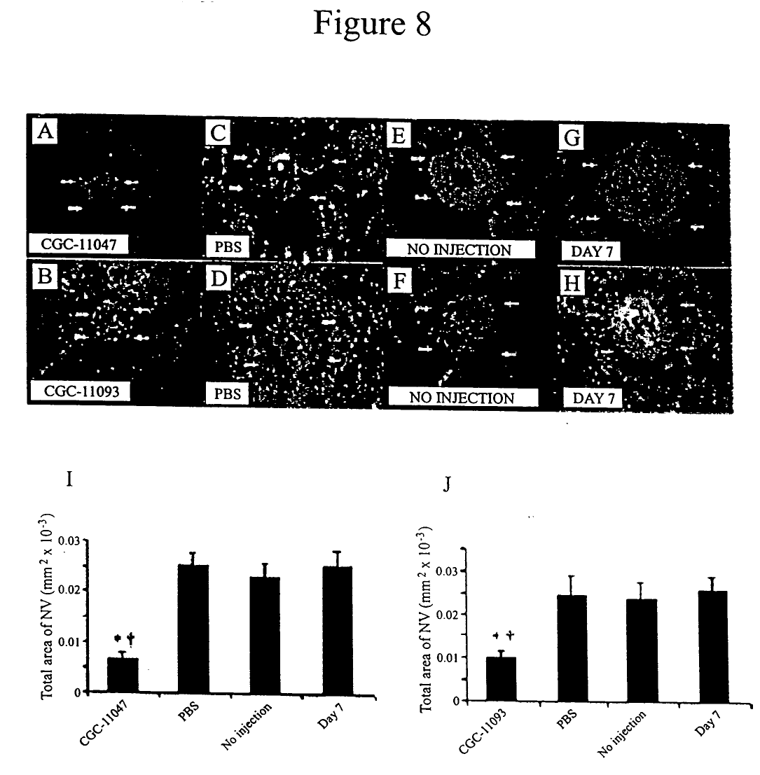

Mice treated with CGC-11047 or CGC-11093 had less CNV than those treated with vehicle and less than the baseline amount of CNV seen at day 7 (FIG. 8).

### Example 11

### Studies on Duration of Effect of a Single Periocular Injection

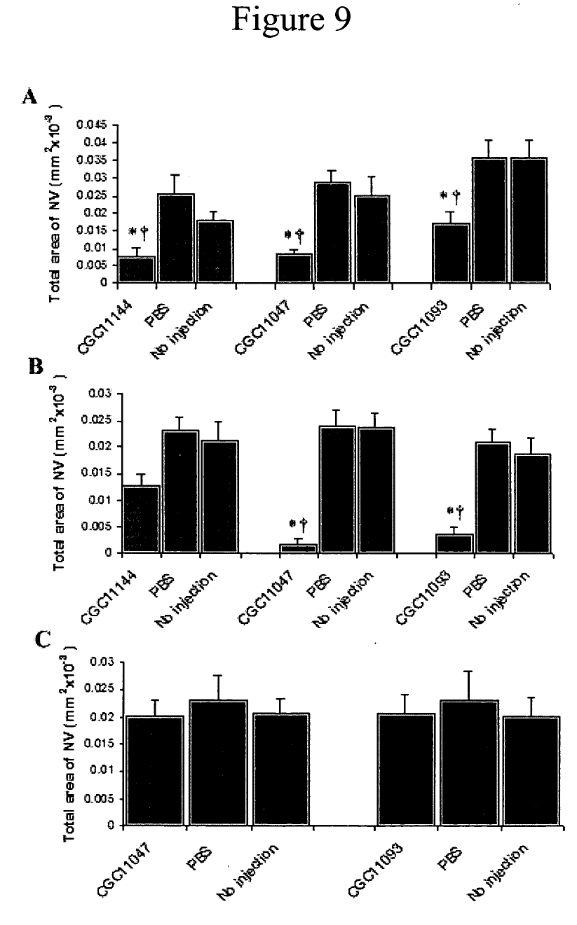

To assess the duration of anti-angiogenic activity from a single periocular injection of polyamine analogs, injections were performed at various time points prior to laser-induced rupture of Bruch's membrane. Compared to injection of vehicle, a single periocular injection of 200 μg of CGC-11144, 2 mg of CGC-11047, or 1.5 mg of CGC-11093 one week prior to rupture of Bruch's membrane resulted in significant reduction of the amount of CNV measured weeks after laser treatment (FIG. 9A). Periocular injection of 2 mg of CGC-11047 or 1.5 mg of CGC-11093 two weeks prior to rupture of Bruch's membrane also resulted in significantly smaller amounts of CNV than the amount present in mice injected with vehicle (FIG. 9B). There was no significant effect from injection of 200 μg of CGC-11144 two weeks prior to rupture of Bruch's membrane (FIG. 9B) nor with injection of 2 mg of CGC-11047 or 1.5 mg of CGC-11093 three weeks before laser treatment (FIG. 9C).

### Example 12

### Oxygen-Induced Ischemic Retinopathy

Ischemic retinopathy can be produced in neonatal C57BL/6 mice as described (Smith, L. E et al., Invest. Opthalmol. Vis. Sci. 35: 101-111 (1994)) and serves as a model for retinopathy of prematurity (ROP). Post-natal day 7 (P7) mice and their mothers were placed in an airtight incubator and exposed to an atmosphere of 75±3% oxygen for 5 days. Incubator temperature was maintained at 23±2° C. and oxygen was continuously monitored with a PROOX model 110 oxygen controller (Reming Bioinstruments Co., Redfield, N.Y.). At post-natal day 12, mice were placed back in room air and were given a periocular injection of 3 μl containing 100 μg of CGC-11144 (n=4), 1 mg of CGC-11047 (n=4), 750 μg of CGC-11093 (n=4) or vehicle. On post-natal day 17 (P17) the treated and control mice were euthanized and the amount of retinal neovascularization was measured.

For measurement of retinal neovascularization, eyes were removed and fixed in 2% paraformaldehyde in phosphate buffered saline (PBS) for one hour and rinsed twice in 25% sucrose in PBS. Specimens were incubated in 25% sucrose in PBS overnight and embedded in OCT compound (Miles Diagnostics, Elkhart, Ind.),). 10 μm frozen sections were post-fixed for 15 minutes in 0.5% glutaraldehyde in PBS and washed in PBS. Sections were also histochemically stained with biotinylated Griffonia simplicifolia lectin B4 (GSA, Vector Laboratories, Burlingame, Calif.), which selectively binds to vascular cells. Slides were incubated in methanol/H₂O₂ for 10 minutes at 4° C., washed with 0.05 M Tris-buffered saline, pH 7.6 (TBS), and incubated for 30 minutes in 10% normal porcine serum. Slides were incubated 2 hours at room temperature with biotinylated GSA and after rinsing with 0.05M TBS, they were incubated with avidin coupled to peroxidase (Vector Laboratories, Burlingame, Calif.) for 30 minutes at room temperature. After being washed for 10 minutes with 0.05M TBS, slides were incubated with diaminobenzidine (Research Genetics, Huntsville, Ala.) to give a brown reaction product.

To perform quantitative assessments, 10 μm frozen serial sections were cut from the iris root on one side of the eye to the iris root on the opposite side of the eye and every tenth section was stained with GSA. Retinas were examined with an Axioskop microscope and images were digitized using a 3 CCD color video camera and a frame grabber. Image-Pro Plus software was used to delineate GSA-stained vascular cells above the internal limiting membrane and the total area of staining was measured.

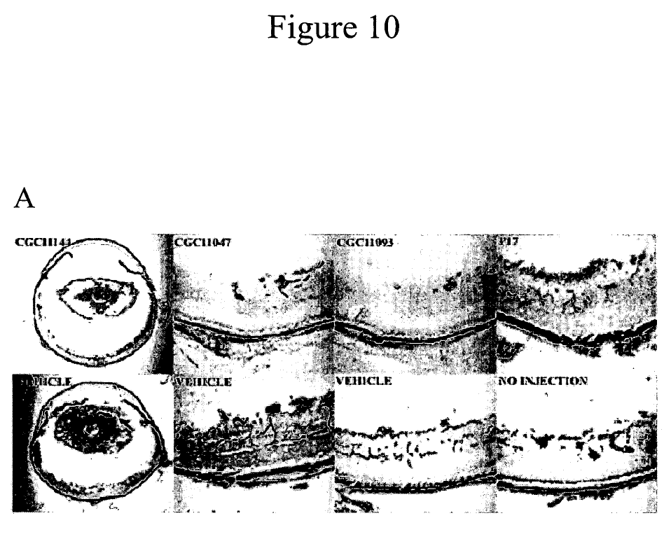

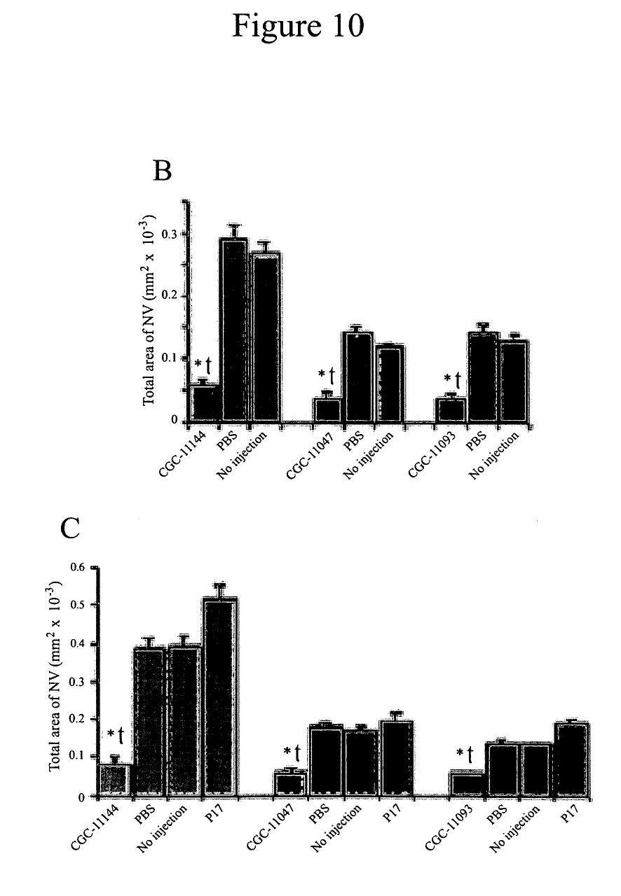

The amount of retinal neovascularization at P17 was significantly less in eyes treated with CGC-11144, CGC-11047 or CGC-11093 as compared to those treated with vehicle (FIGS. 10A and 10C).

To investigate the effect of these agents on regression of retinal neovascularization, ischemic retinopathy was induced and left untreated until P17, when the baseline amount of retinal neovascularization was measured in one group of mice. From post-natal days 17 to 20 (P17 to P20) the remainder of the mice were given daily periocular injections of 100 μg of CGC-11144 (n=4), 1 mg of CGC-11047 (n=4), 750 μg of CGC-11093 (n=4) or vehicle. On P20 the mice were euthanized and the amount of retinal neovascularization in the treated and control mice was measured as described above. At P20, mice that had been treated with CGC-11144, CGC-11047 or CGC-11093 had significantly less neovascularization than those that had been treated with vehicle (FIG. 10B).

### Example 13

### Studies in Rhodopsin/VEGF Transgenic Mice

Transgenic mice (V6 mice) in which the rhodopsin promoter drives expression of VEGF₁₆₅ in photoreceptors have onset of VEGF expression at post-natal day 7 (P7) and soon after begin to develop neovascularization originating from the deep capillary bed that grows through the photoreceptor layer into the subretinal space. (Okamoto N et al., Amer. J. Pathol. 151: 281-91 (1997); Tobe T et al., Invest. Ophthalmol. Vis. Sci. 39: 180-188 (1998)). Hemizygous transgene positive mice were given a periocular injection of 100 μg of CGC-11144 (n=3), 1 mg of CGC-11047 (n=3), 750 μg of CGC-11093 (n=3) or vehicle (n=7) at P7. At post-natal day 21 (P21), mice were euthanized and the amount of subretinal neovascularization was quantified as previously described (Tobe, ibid.). Briefly, mice were anesthetized, perfused with fluorescein-labeled dextran, and the number of neovascular lesions on the outer surface of the retina and their total area were measured on retinal flat mounts by image analysis as described in Example 1.

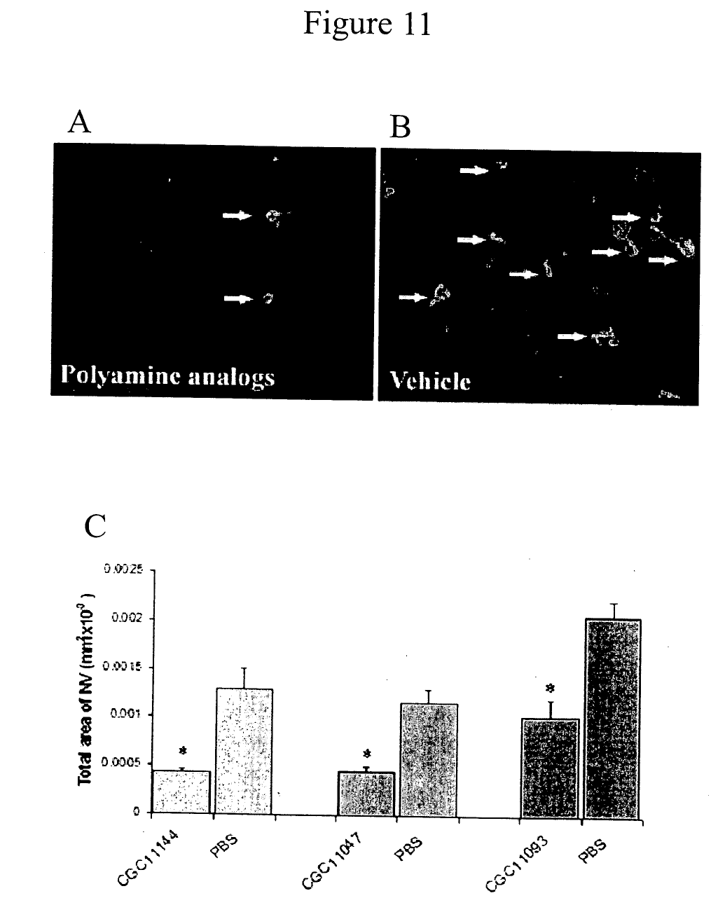

Measurement of the subretinal neovascularization by image analysis showed that on day 14 mice treated with periocular. CGC-11144, CGC-11047 or CGC-11093 had significantly less subretinal neovascularization than eyes treated with vehicle (FIG. 11).

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of treating ocular disease, comprising:
administering one or more conformationally restricted polyamine analogs to a subject with an ocular disease in a therapeutically effective amount on the ocular disease, wherein the ocular disease is characterized by undesirable cell proliferation or neovascularization;
with the proviso that the conformationally restricted polyamine analog is not a macrocyclic polyamine analog.

2. The method of claim 1, wherein the ocular disease is characterized by neovascularization.

3. The method of claim 1, wherein the ocular disease is characterized by retinal neovascularization.

4. The method of claim 1, wherein the ocular disease is characterized by choroidal neovascularization.

5. The method of claim 1, wherein the ocular disease is macular degeneration.

6. The method of claim 5, wherein the ocular disease is dry macular degeneration.

7. The method of claim 5, wherein the ocular disease is wet macular degeneration.

8. The method of claim 5, wherein the conformationally restricted polyamine analog is 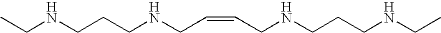
or any stereoisomer, salt, hydrate, or solvate thereof.

9. The method of claim 7, wherein the conformationally restricted polyamine analog is 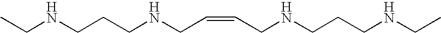
or any stereoisomer, salt, hydrate, or solvate thereof.

10. The method of claim 1, wherein the one or more conformationally restricted polyamine analogs is administered in an amount sufficient to reduce retinal neovascularization, to delay the development of retinal neovascularization, to prevent the development of retinal neovascularization, or to cause regression of retinal neovascularization.

11. The method of claim 1, wherein the one or more conformationally restricted polyamine analogs is administered in an amount sufficient to reduce choroidal neovascularization, to delay the development of choroidal neovascularization, to prevent the development of choroidal neovascularization, or to cause regression of choroidal neovascularization.

12. The method of claim 1, wherein the one or more conformationally restricted polyamine analogs is selected from the group consisting of 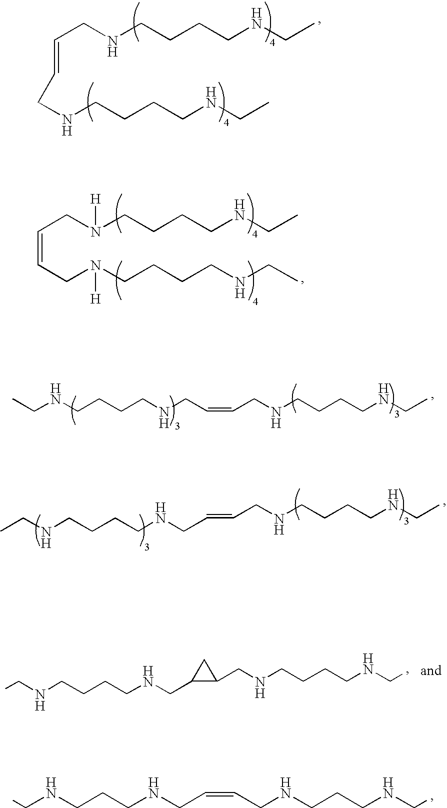
and all stereoisomers, salts, hydrates, and solvates thereof.

13. The method of claim 1, wherein the one or more conformationally restricted polyamine analogs is administered in a composition suitable for ophthalmic administration.

14. The method of claim 13, wherein the one or more conformationally restricted polyamine analogs is selected from the group consisting of 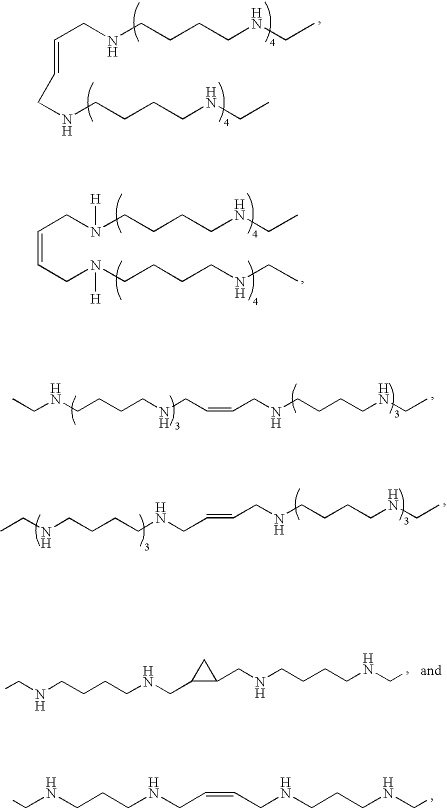
and all stereoisomers, salts, hydrates, and solvates thereof.

15. The method of claim 1, wherein the one or more conformationally restricted polyamine analogs is administered via periocular administration.

16. The method of claim 15, wherein the periocular administration is periocular injection.

17. The method of claim 1, wherein the one or more conformationally restricted polyamine analogs is administered via intravitreous administration.

18. The method of claim 17, wherein the intravitreous administration is intravitreous injection.

19. The method of claim 1, wherein the one or more conformationally restricted polyamine analogs is administered via a sustained release formulation, a sustained release implant, or a sustained release device.

20. The method of claim 19, wherein the sustained release formulation, sustained release implant, or sustained release device is placed in the periocular tissue.

21. The method of claim 19, wherein the sustained release formulation, sustained release implant, or sustained release device is placed in the vitreous chamber or vitreous body.

22. The method of claim 1, wherein the one or more conformationally restricted polyamine analogs is administered at a frequency of about once a week for about two months to about twelve months, about once every two weeks for about two months to about twelve months, about once every three weeks for about two months to about twelve months, or about once every four weeks for about two months to about twelve months.

23. The method of claim 22, wherein the one or more conformationally restricted polyamine analogs is administered via periocular administration.

24. The method of claim 23, wherein the periocular administration is periocular injection.

25. The method of claim 1, wherein the one or more conformationally restricted polyamine analogs is administered in an ophthalmic formulation.

26. The method of claim 25, wherein the ophthalmic formulation comprises a buffer system selected from the group comprising sodium phosphate, sodium acetate, sodium citrate or sodium borate.

27. The method of claim 25, wherein the ophthalmic formulation comprises a physiologically balanced irrigating solution.

28. A composition comprising a conformationally restricted polyamine analog and a pharmaceutical carrier suitable for ophthalmic administration.

29. The composition of claim 28, wherein the composition is suitable for periocular administration.

30. The composition of claim 29, wherein the composition is suitable for periocular injection.

31. A composition or device comprising a conformationally restricted polyamine analog in a sustained release formulation or sustained release device.

32. The composition or device of claim 31, wherein the sustained release formulation or sustained release device is suitable for ophthalmic administration.

33. The composition or device of claim 32, wherein the sustained release formulation or sustained release device is suitable for periocular administration.

34. The method of claim 1, wherein the conformationally restricted polyamine analog is selected from the group of compounds of the formula
E-NH-D-NH—B-A-B—NH-D-NH-E
wherein A is independently selected from the group consisting of C₂-C₆ alkene and C₃-C₆ cycloalkyl, cycloalkenyl, and cycloaryl; B is independently selected from the group consisting of a single bond and C₁-C₆ alkyl and alkenyl; D is independently selected from the group consisting of C₁-C₆ alkyl and alkenyl, and C₃-C₆ cycloalkyl, cycloalkenyl, and cycloaryl; E is independently selected from the group consisting of H, C₁-C₆ alkyl and alkenyl; and all salts, hydrates, solvates, and stereoisomers thereof.
